(19) United States
(12) Patent Application Publication
(10) Pub. No.: US 2014/0155635 A1
(43) Pub. Date: Jun. 5, 2014

(54) RESORCYLIC ACID LACTONE COMPOUNDS

(71) Applicant: KOREA INSTITUTE OF SCIENCE AND TECHNOLOGY, Seoul (KR)

(72) Inventors: Tae Bo SIM, Daegu (KR); Ho Jong Yoon, Seoul (KR); Ji Nyeo Kim, Seoul (KR)

(73) Assignee: KOREA INSTITUTE OF SCIENCE AND TECHNOLOGY, Seoul (KR)

(21) Appl. No.: 14/066,053

(22) Filed: Oct. 29, 2013

(30) Foreign Application Priority Data
Nov. 20, 2012 (KR) 10-2012-0131915

(51) Int. Cl. C07D 313/00; C07D 493/08; C07D 317/30; C07D 407/10; C07D 319/08; C07D 317/32

(52) U.S. Cl. CPC C07D 313/00; C07D 319/08; C07D 317/32; C07D 317/30; C07D 407/10; C07D 493/08
USPC 549/268; 549/274; 549/450; 549/454

(57) ABSTRACT

Disclosed are a novel resorcyclic acid lactone compound with inhibitory activity against protein kinases, a pharmaceutically acceptable salt thereof, a method for the synthesis thereof, and a pharmaceutical composition for the treatment and prevention of various cancer diseases comprising the same as an active ingredient. The novel resorcyclic acid lactone compound is useful as a therapeutic for cancer diseases, especially blood cancer, inter alia, acute myeloid leukemia (AML).

### CROSS-REFERENCE TO RELATED APPLICATIONS

This application claims priority under 35 U.S.C. §119 to Korean Patent Application No. 10-2012-0131915, filed on Nov. 20, 2012, in the Korean Intellectual Property Office, the disclosure of which is incorporated herein by reference in its entirety.

### BACKGROUND OF THE INVENTION

1. Field of the Invention

The present invention relates to a novel resorcyclic acid lactone compound with inhibitory activity against protein kinase, a pharmaceutically acceptable salt thereof, a method for preparing the same, and a pharmaceutical composition for the treatment and prevention of various cancer diseases, comprising the same as an active ingredient.

2. Description of the Related Art

L-783277 is a naturally occurring material isolated from the fruit body Helvella acetabulum. Since the first report on the de novo synthesis of the natural material in 2008, many studies have been focused on the improvement in the synthesis thereof.

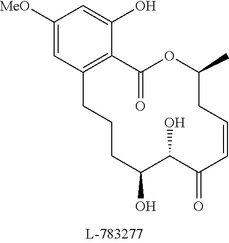 [SMILES: COc1cc(O)c2c(c1)CCC[C@H](O)[C@H](O)C(=O)/C=C\C[C@H](C)OC2=O]

L-783277 is a 14-membered macrocyclic compound based on a resorcinol carboxylate and cis-enone structure, with an anti-diol moiety in the microcycle. L-783277 is an irreversible ATP competitive inhibitor against the serine/threonine kinase MAPK (mitogen-activated protein kinase), and exhibits weak inhibitory activity against LCK (lymphocyte-specipic protein tyrosine kinase).

On the whole, resorcylic acid lactones (hereinafter, referred to as “RALs”) are known to inhibit the MAP signaling pathway comprised of four cascade levels including mitogen receptors, MEK 1/2, ERK 1/2, and sub-ERK. Out of approximately 518 kinases, as many as 46 including FLT (VEGFR 1), MEK 1/2 are within the inhibition coverage of RALs. Sharing the feature of RALs, L-783277 are also found to have potent inhibitory activity against various kinases including VEGFR 2 and FLT 3/4 as well as MEK 1/2, which is presumably due to the presence of cis-enone structure. The cis-enone structure is essential for the activity of RALs. The cis-enone structure of RALs interacts, via 1,4-addition reaction, with the cystein residue (Cys) in the active site of kinases at which the Cys binds to ATP, thus acting as a selevive, irreversible kinase inhibitor. That is, the cis-enone structure confers on the compound the selectivity for the 46 kinases which contain the Cys residue, whereas the inhibitory activity of the compound is not observed in almost all of the kinases which are devoid of the Cys residue.

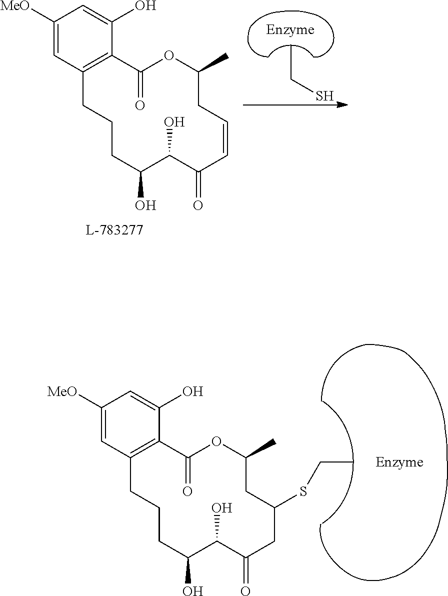

Although RALs with a cis-enone structure have selective inhibition against the kinases which contain the Cys residue, L-783277 is not said to be of high selectivity for kinases, that is, to highly selectively act on desired kinase targets.

A recent report showed the synthesis of various derivatives of L-783277, together with the comparison of their activities [Liniger, M.; Altmann, K. H. ACS Med. Chem. Lett., 2011, 2, 22-27].

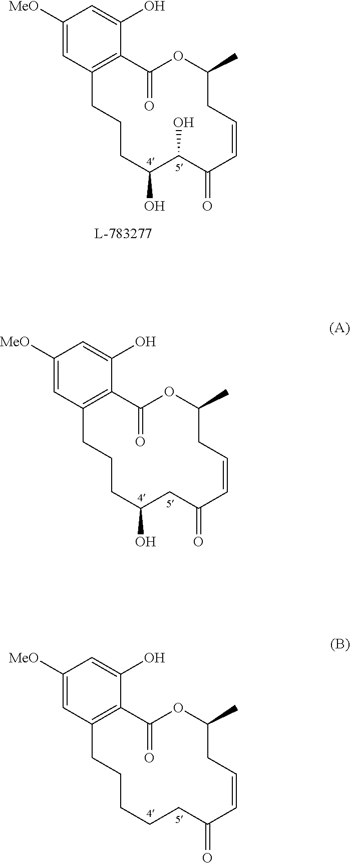 [SMILES: COc1cc(O)c2c(c1)CCC[C@H](O)[C@H](O)C(=O)/C=C\C[C@H](C)OC2=O ; COc1cc(O)c2c(c1)CCC[C@H](O)CC(=O)/C=C\C[C@H](C)OC2=O ; COc1cc(O)c2c(c1)CCCCCC(=O)/C=C\C[C@H](C)OC2=O]

The resorcyclic acid lactone compound which the present invention features is a novel compound which has not been reported in any literature prior to the present invention, and exhibits potent inhibitory activity with high selectivity for Cys-containing protein kinases.

### SUMMARY OF THE INVENTION

It is therefore an object of the present invention to provide a novel resorcyclic acid lactone compound with excellence in inhibitory activity and selectivity for protein kinases.

It is another object of the present invention to provide a pharmaceutical composition for the prevention and treatment of cancer, comprising the novel compound or a pharmaceutically acceptable salt thereof as an active ingredient.

It is a further object of the present invention to provide a method for the preparation of the novel compound.

It is a still further object of the present invention to provide an intermediate compound suitable for use in the synthesis of the novel compound.

In order to accomplish the above objects, the present invention provides at least one selected from the group consisting of a resorcyclic acid lactone compound represented by the following Chemical Formula F, a pharmaceutically acceptable salt thereof, a hydrate thereof, or a solvate thereof:

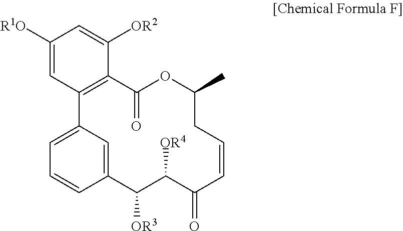

wherein R¹, R², R³, and R⁴ independently represent a hydrogen atom, or C1-10 alkyl.

Containing two or more chiral centers therein, the resorcyclic acid lactone compound of Chemical Formula F may exist in the form of respective enantiomers or diastereomers, or in a racemic mixture.

Having potent inhibitory activity against protein kinases including FLT3 (D835Y), FLT3 (ITD), FLT3, FLT1/VEGFR1, FLT4/VEGFR3, PDGFRa, PDGFRb, PDGFRa (D842V), PDGFRa (T674I), and PDGFRa (V561D), the resorcyclic acid lactone compound of the present invention may be used as an active ingredient in a pharmaceutical composition for the prevention and treatment of cancer diseases.

The pharmaceutical composition of the present invention exhibits a high therapeutic effect on blood cancer, particularly, acute myeloid leukemia (AML).

### DESCRIPTION OF THE PREFERRED EMBODIMENTS

Pharmaceutically acceptable salts of the compound of Chemical Formula F according to the present invention may be prepared using a method typical in the art. A pharmaceutically acceptable salt should be of low toxicity to the human body, without degradation in the biological activity and physicochemical properties of the parent compound from which it is derived. Among the pharmaceutically acceptable salts of the compound of Chemical Formula F may be acid addition salts of pharmaceutically acceptable free acids and alkaline compounds of Chemical Formula F, alkali metal salts (sodium, etc.), alkaline earth metal salts (potassium, etc.), organic base addition salts of the carboxylic acid of Chemical Formula F, and amino acid addition salts. Free acids available for the preparation of pharmaceutically acceptable salts may be organic or inorganic. Examples of the inorganic acid include hydrochloric acid, sulfuric acid, nitric acid, phosphoric acid, perchloric acid, hydrobromic acid. Examples of the organic acid include acetic acid, methane sulfonic acid, ethane sulfonic acid, p-toluene sulfonic acid, furmaric acid, maleic acid, malonic acid, phthalic acid, succinic acid, lactic acid, citric acid, gluconic acid, tartaric acid, salicylic acid, fumaric acid, malic acid, oxalic acid, benzoic acid, embonic acid, aspartic acid, and glutamic acid. Tris(hydroxymethyl)methylamine and dicyclohexylamine may be available as organic bases for the preparation of organic base addition salts. Natural amino acids such as alanine, glycine, etc. are suitable for use in the preparation of amino acid addition bases.

In addition to the compounds represented by Chemical Formula F, pharmaceutically acceptable salts thereof, hydrates thereof, and solvates thereof fall within the scope of the present invention. The pharmaceutically acceptable salts may be prepared using a conventional method as described below. Briefly, a base compound of Chemical Formula F is dissolved in a water-miscible solvent such as methanol, ethanol, acetone, 1,4-dioxane, etc. and mixed with a free acid or free base, followed by crystallization or recrystallization.

Existence of one or more asymmetric carbons renders the compound of Chemical Formula F into enantiomers or diastereomers. Accordingly, each isomer or a mixture of isomers is within the scope of the present invention. Different isomers can be separated or dissolved by typical methods. Alternatively, a desired isomer may be obtained using a synthesis method or through stereospecific or asymmetric synthesis.

Also, the present invention speculates a radioactive derivative of the compound of Chemical Formula F, which is useful in the biotrepy field.

The term “alkyl,” used as substituents to define the compound of the present invention, refers to an aliphatic saturated hydrocarbon containing 1 to 10 carbon atoms, as exemplified by methyl, ethyl, n-propyl, i-propyl, n-butyl, i-butyl, t-butyl, n-pentyl, i-pentyl, neopentyl, t-pentyl, n-hexyl, i-hexyl, heptyl, octyl, etc.

In a preferred embodiment of the compound of Chemical Formula F, R⁴ is C1˜C10 alkyl, and R³ and R⁴ are each a hydrogen atom.

In a further embodiment, the compound of Chemical Formula F is (7S,12S,13R,Z)-4,12,13-trihydroxy-2-methoxy-7-methyl-7,8,13,14-tetrahydro-5H-dibenzo[c,e][1]oxacyclotetradecen-5,11(12H)-dione.

Also contemplated in accordance with another embodiment of the present invention is a pharmaceutical composition comprising the compound of Chemical Formula F, a pharmaceutically acceptable salt thereof, a solvate thereof, or a hydrate thereof as an active ingredient.

Exhibiting potent inhibitory activity against protein kineases, the compound of Chemical Formula F is applicable as a protective or therapeutic for diseases characterized by abnormal cell growth.

Among the protein kinases which can be inhibited by the compound of Chemical Formula F are FLT3 (D835Y), FLT3 (ITD), FLT3, FLT1/VEGFR1, FLT4/VEGFR3, PDGFRa, PDGFRb, PDGFRa (D842V), PDGFRa (T674I), and PDGFRa (V561D).

The diseases caused by abnormal cell growth are typically cancer examples of which include stomach cancer, lung cancer, colorectal cancer, small intestine cancer, pancreatic cancer, brain cancer, bone cancer, melanoma, breast cancer, sclerosing adenosis, uterine cancer, uterine cervical cancer, head and neck cancer, esophageal cancer, thyroid cancer, parathyroid cancer, renal cancer, sarcoma, prostate cancer, urethral cancer, bladder cancer, blood cancer, such as leukemia, multiple myeloma, and myelodysplastic syndrome, lymphoma such as Hodgkin lymphoma and non-Hodgkin lymphoma, and fibroadenoma. The pharmaceutical composition of the present invention is useful for preventing and treating cancer diseases, especially blood cancer, inter alia, acute myeloid leukemia (AML).

The pharmaceutical composition of the present invention comprises the compound of Chemical Formula F, a pharmaceutically acceptable salt thereof, a solvate thereof, or a hydrate thereof as an active ingredient and may be formulated, in combination with a pharmaceutically acceptable additive, such as a carrier, a supplement and/or an excipient, into a typical formulation, for example, an oral dosage form, such as a tablet, a capsule, a troche, a liquid, a suspension, etc., or a non-oral dosage form.

As excipients in the pharmaceutical composition of the prenset invention, sweeteners, binders, solubilizers, dissolution aids, humectants, emulsifiers, isotonics, absorbers, disintegrants, antioxidants, preservatives, lubricants, fillers, and aromatics may be available. Examples of the excipients include lactose, dextrose, sucrose, mannitol, sorbitol, cellulose, glycine, silica, talc, stearic acid, sterin, magnesium stearate, magnesium aluminum silicate, starch, gelatin, tragacanth gum, alginic acid, sodium alginate, methyl cellulose, sodium carboxyl methyl cellulose, agar, water, ethanol, polyethylene glycol, polyvinylpyrrolidone, sodium chloride, calcium chloride, orange essence, strawberry essence, and vanilla perfume.

The effective dosage of the compound in accordance with the present invention depends on various factors, including the patient's age, weight, gender, the route of administration, the state of health, the severity of diseases, etc. Typically, the compound according to the present invention may be administered at a daily dose ranging from 0.01 to 1,000 mg for 70 kg adult patients. The compound may be administered in a single dose or may be divided into multiple doses per day according to the instructions of a physician or pharmacist.

In another embodiment thereof, the present invention addresses a method for synthesizing the compound of Chemical Formula F. The compound of Chemical Formula F in accordance with the present invention may be synthesized through a series of processes illustrated in the following Reaction Schemes 1 to 4. The synthesis method of the present invention will be delineated with reference to the following Reaction Schemes.

In the following Reaction Scheme 1, three reaction steps are carried out:

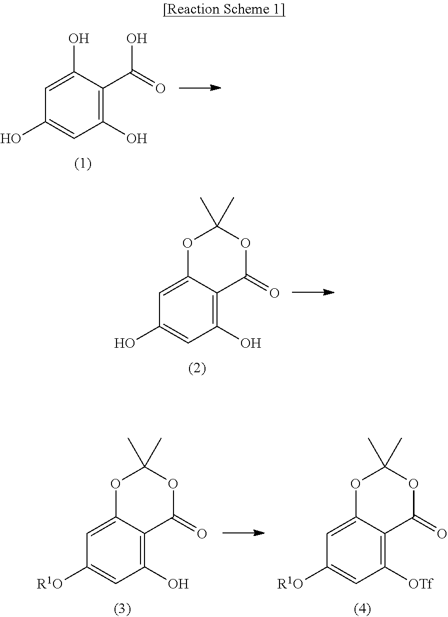

wherein R¹ represents C1˜C10 alkyl.

In Reaction Scheme 1, first, a protection group is introduced into 2,4,6-trihydroxybenzoic acid compound (1) by reaction with trifluoroacetic acid (TFA) and trifluoroacetic anhydride (TFAA) at 30° C. to 80° C. in acetone to give compound (2). Subsequently, compound (2) is subjected to the Mitsunobu reaction to introduce, for example, a methyl group thereinto. The resulting compound (3) is protected with trifluoromethanesulfonate (OTf) at the hydroxy group of the phenol moiety to affod compound (4).

Starting with compound (4), the following Reaction Scheme 2 is carried out in a five-step process:

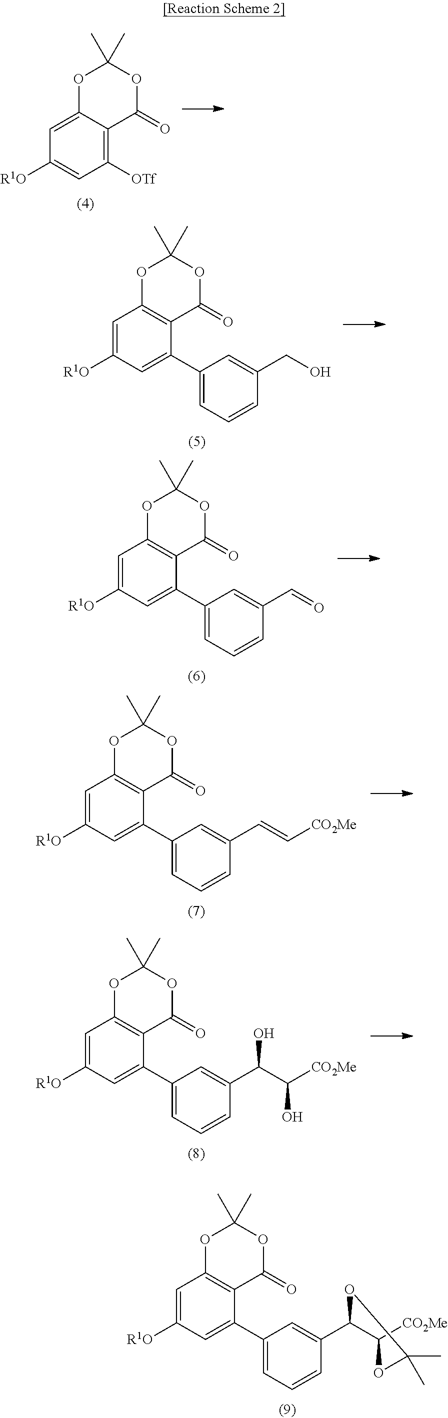

wherein R¹ represents C1˜C10 alkyl.

In Reaction Scheme 2, the phenyltrifluoromethanesulfonate compound (4) is first converted into an alcohol compound (5) by the Suzuki coupling in the presence of a bronic acid compound. The next step is to oxidize the alcohol compound (5) into an aldehyde compound (6) in the presence of MnO₂. Subsequently, the aldehyde (6) is reacted with methyl(triphenylphosphoranylidene)acetate to give an ester compound (7), with a double bond newly introduced thereinto. Then, the ester compound (7) is allowed to undergo dehydroxylation with methanesulfone amide and AD-mix at the double bond to produce a dihydroxy compound (8). Finally, the dihydroxy compound (8) is protected with 2,2-dimethoxypropane and pyridinium p-toluenesulfonate (PPTS) to give a protected compound (9).

Reaction Scheme 3 utilizes compound (9) as a starting material and is carried out in a three-step process:

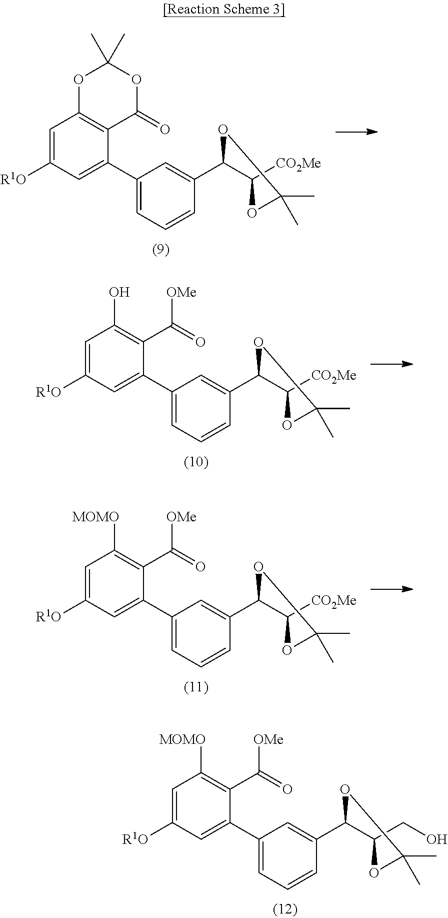

wherein R¹ represents C1˜C10 alkyl and MOM represents methoxymethyl.

As can be seen, 2-hydroxy benzoic acid of compound (9) is deprotected by reacting with sodium methoxide (NaOMe), and then with methyl iodide (MeI) in the presence of potassium hydrogen carbonate to give compound (10) into which the protecting group methoxymethyl (MOM) is then introduced. Finally, compound (11) is reduced at the ester group into compound (12).

The following Reaction Scheme 4 is carried out in a 4-step process, staring from compound (12):

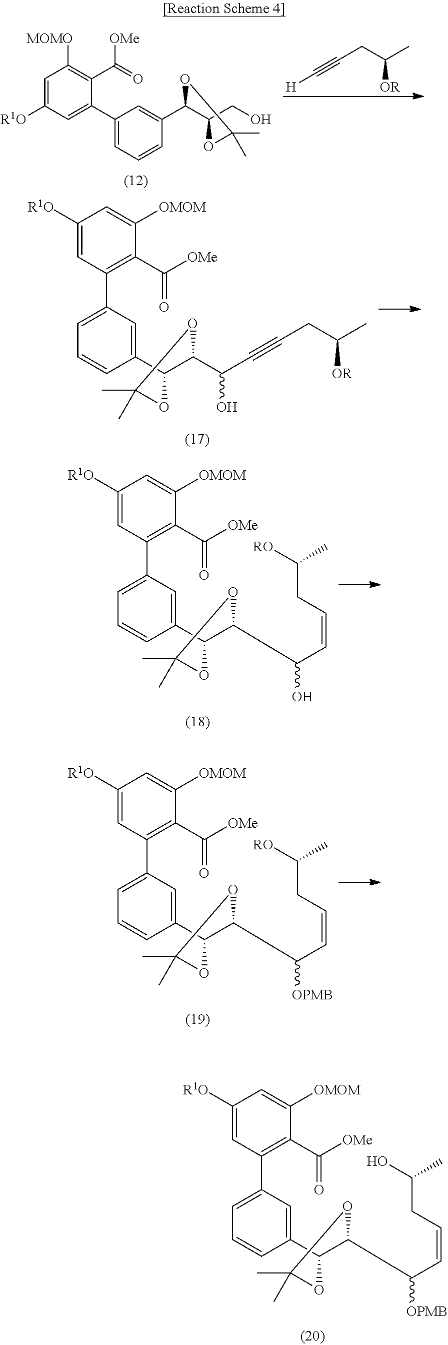

wherein R¹ represents C1˜C10 alkyl, R represents tert-butyldiphenylsilyl (-TBDPS) or tert-butyldimethylsilyl (-TBS), MOM represents methoxymethyl, and PMB represents p-methoxybenzyl.

In Reaction Scheme 4, compound (12) is oxidized with Dess-Martin periodianane in the presence of a base and then reacted with (R)-tert-butyl(pent-4-yn-2-yloxy)diphenylsilane or (R)-tert-butyldimethyl(pent-4-yn-2-yloxy)silane to give an acetylene-incorporated compound 17 protected with tert-butyldiphenylsilane (TBDPS) or tert-butyldimethylsilane (TBS). In the next step, the acetylene of compound 17 is reduced into ethylene by reaction with quinoline in the presence of the Lindlar catalyst Pd—BaSO₄ in a hydrogen atmosphere to affod compound (18). Subsequently, the compound 18 is protected with p-methoxybenzyl (PMB) from p-methoxybenzyl chloride (PMBC1) in the presence of NaH and NaI to give compound (19). From compound (19), tert-butyldiphenylsilane (TBDPS) or tert-butyldimethylsilane (TBS) is allowed to leave, resulting in a deprotected compound (20).

Reaction Scheme 5 is a 4-step process with compound (20) serving as a starting material:

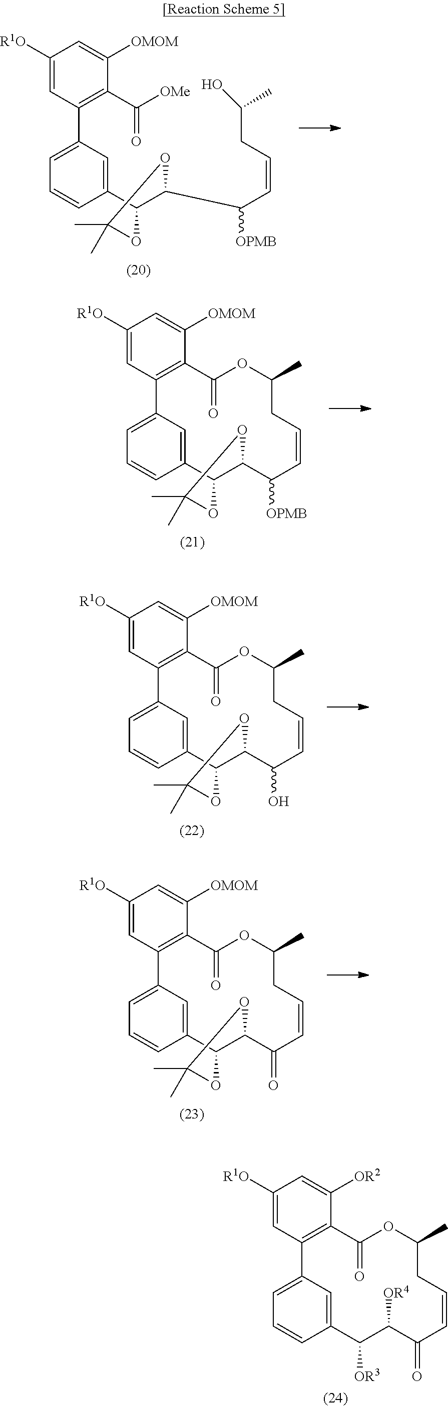

wherein R¹, R², R³, and R⁴ are each independently a hydrogen atom or C1˜C10 alkyl, MOM represents methoxymethyl, and PMB represents p-methoxybenzyl.

As illustrated in Reaction Scheme 5, compound (20) is converted into a lactam compound (21) through hydrolysis and Mitsunobu reaction. Briefly, compound (20) is heated at a pH of 7 to 10, and a temperature of 60 to 90° C., followed by cyclization with triphenylphephine at −10 to 30° C. in the presence of diisopropyl azodicarboxylate to give a lactam compound (21). Subsequently, compound (21) is converted into an alcohol compound (22) by deprotecting p-methoxybenzyl (PMB) in the presence of 2,3-dichloro-5,6-dicyano-1,4-benzoquinone. The next step is Dess-Martin periodianane oxidation in the presence of a base to produce an α,β-unsaturated ketone compound (23). Finally, the protecting group methoxymethyl(MOM) is removed from compound (23) using trifluoroacetic acid to produce a resorcyclic acid lactone compound (24).

For use in the processes of Reaction Schemes 1 to 5, an organic solvent may be selected from among tetrahydrofuran, acetone, dichloromethane, dimethylformamide, methanol, ethylacetate, and a combination thereof. The inorganic base may be an alkali metal salt or an alkaline earth metal salt, as concretely exemplified by KHCO₃, K₂CO₃, NaHCO₃, and Na₂CO₃. As an organic base, triethylamine and/or pyridine may be used.

On the other hand, the TBDPS- or TBS-protected acetylene compound (16) used in the first step of Reaction Scheme 4 may be synthesized, as illustrated in Reaction Scheme 6 below:

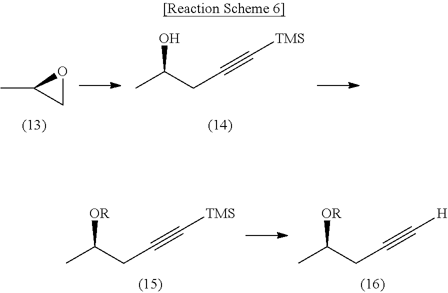

wherein R represents tert-butyldiphenylsilyl (-TBDPS) or tert-butyldimethylsilyl (-TBS), and TMS represents trimethylsilyl.

According to Reaction Scheme 6, the epoxide ring of (R)-2-methyloxirane (13) is opened by reaction with ethynyltrimethyl silane in the presence of BF₃-diethylether and n-butyl lithium to give an acetylene compound (14) which is then reacted with (R)-tert-butyl(pent-4-yn-2-yloxy)diphenylsilane or (R)-tert-butyldimethyl(pent-4-yn-2-yloxy)silane, together with imidazole. From the resulting acetylene compound (15) protected with tert-butyldiphenylsilane (TBDPS) or tert-butyldimethylsilane (TBS), the trimethylsilane (TMS) is removed to produce compound (16).

Although a description in Reaction Schemes 1 to 6 is limited to isomers of specific stereostructures, it should be understood that isomers with inverse configurations, or racemates can be readily synthesized.

Some of the intermediate compounds synthesized during the processes of Reaction Schemes 1 to 6 are novel compounds. Accordingly, the present invention envisages the novel intermediate compounds, as well.

Concrete examples of the novel intermediate compounds of the present invention include, but are not limited to:
5-(3-(Hydroxymethyl)phenyl)-7-methoxy-2,2-dimethyl-4H-benzo[d][1,3]dioxin-4-one);
3-(7-Methoxy-2,2-dimethyl-4-oxo-4H-benzo[d][1,3]dioxin-5-yl)benzaldehyde;
(E)-Methyl 3-(3-(7-methoxy-2,2-dimethyl-4-oxo-4H-benzo[d][1,3]dioxin-5-yl)phenyl)acrylate;
(2S,3R)-methyl 2,3-dihydroxy-3-(3-(7-methoxy-2,2-dimethyl-4-oxo-4H-benzo[d][1,3]dioxin-5-yl)phenyl)propanoate;
(4S,5R)-methyl 5-(3-(7-methoxy-2,2-dimethyl-4-oxo-4H-benzo[d][1,3]dioxin-5-yl)phenyl)-2,2-dimethyl-1,3-dioxorane-4-carboxylate;
(4S,5R)-methyl 5-(3′-hydroxy-5′-methoxy-2′-(methoxycarbonyl)-[1,1′-byphenyl]-3-yl)-2,2-dimethyl-1,3-M dioxorane-4-carboxylate;
(4S,5R)-methyl 5-(5′-methoxy-2′-(methoxycarbonyl)-3′-(methoxymethoxy)-[1,1′-biphenyl]-3-yl)-2,2-dimethyl-1,3-dioxorane-4-carboxylate;
methyl 3′-((4R,5R)-5-(hydroxymethyl)-2,2-dimethyl-1,3-dioxoran-4-yl)-5-methoxy-3-(methoxymethoxy)-[1,1′-biphenyl]-2-carboxylate;
methyl 3′-(4R,5R)-5-((5R)-5-(tert-butyldiphenylsilyl)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate;
methyl 3′-(4R,5R)-5-((5R)-5-((tert-butyldimethylsilyl)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate;
methyl 3′-((4R,5R)-5-((5R,Z)-5-((tert-butyldiphenylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate;
methyl 3′-((4R,5R)-5-((5R,Z)-5-((tert-butyldimethylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate;
methyl 3′-((4R,5R)-5-((5R,Z)-5-((tert-butyldiphenylsilyl)oxy)-1-((4-methoxybenzyl)oxy)hex-2-en-1-M yl)-2,2-dimethyl-1,4-dioxoran-4-yl)-5-methoxy-3-(methoxymethoxy)-[1,1′-biphenyl]-2-carboxylate;
methyl 3′-((4R,5R)-5-((5R,Z)-5-((tert-butyldimethylsilyl)oxy)-1-((4-methoxy benzyl)oxy)hex-2-en-2-yl)-2,2-dimethyl-1,3-dioxoran-4-yl)-5-methoxy-3-(methoxymethoxy)-[1,1′-biphenyl]-2-carboxylate;
methyl 3′-((4R,5R)-5-((5R,Z)-5-hydroxy-1-((4-methoxybenzyl)oxy)hex-2-en-1-yl)-2,2-dimethyl-1,3-dioxoran-4-yl)-5-methoxy-3-(methoxymethoxy)-[1,1′-biphenyl]-2-carboxylate;
(3aR,8S,19aR,Z)-13-methoxy-4-((4-methoxybenzyl)oxy)-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one;
(3aR,8S,19aR,Z)-4-hydroxy-13-methoxy-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one; and
(3aS,8S,19aR,Z)-13-methoxy-11-(methoxymethoxy)-2,2,8-trimethyl-7,8,19,19a-tetrahydro-3aH-dibenzo[c,e][1.3]dioxolo[4,5-h][1]oxacyclotetradecen-4,10-dione.

A better understanding of the present invention may be obtained through the following examples which are set forth to illustrate, but are not to be construed as limiting the present invention.

### SYNTHESIS EXAMPLES

### Example 1

### Synthesis of 7-Methoxy-2,2-dimethyl-4-oxo-4H-benzo[d][1.3]dioxin-5-yltrifluoromethanesulfonate (Compound 4)

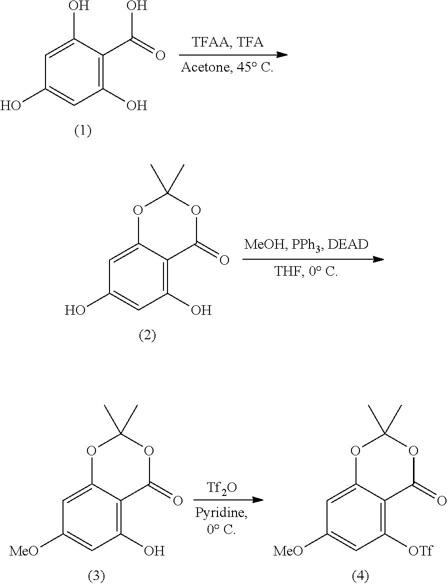 [SMILES: "Tf2O" ; O=C(O)c1c(O)cc(O)cc1O ; CC1(C)OC(=O)c2c(O)cc(O)cc2O1 ; COc1cc(O)c2c(c1)OC(C)(C)OC2=O ; COc1cc2c(c(OS(=O)(=O)C(F)(F)F)c1)C(=O)OC(C)(C)O2]

### Step 1. 5,7-Dihydroxy-2,2-dimethyl-4H-benzo[d][1,3]dioxin-4-one (Compound 1)

To a solution of 2,4,6-trihydroxybenzoic acid (50 g, 294.08 mmol) in acetone (145 mL, 1.97 mol) was added trifluoroacetic acid (210 mL, 2.94 mol) at 45° C. while stirring for 1 hr. Then, trifluoroacetic acid (125 mL, 8.93 mol) was added to the reaction mixture at the same temperature. The reaction mixture was stirred for 3 hrs at 45° C. After completion of the reaction, the solvent was evaporated at reduced pressure. The residue was diluted in methylene chloride, and washed with water and then with a saturated sodium hydrogen carbonate solution. The pooled organic layers were dried over anhydrous magnesium sulfate, filtered, and concentrated at reduced pressure. The residue was recrystallized in ethanol to obtain the title compound 5,7-dihydroxy-2,2-dimethyl-4H-benzo[d][1,3]dioxin-4-one as a white solid (24 g, 39% yield). The mother liquid was purified by column chromatography (silica gel, DCM:MeOH=99:1→97:3) to further produce the title compound 5,7-dihydroxy-2,2-dimethyl-4H-benzo[d][1,3]dioxin-4-one (11 g, 18% yield).

¹H NMR (300 MHz, CDCl₃) δ 10.46 (s, 1H), 6.08 (d, J=2.2 Hz, 1H), 5.95 (d, J=2.2 Hz, 1H), 5.44 (s, 1H), 1.74 (s, 6H).

### Step 2. 5-Hydroxy-7-methoxy-2,2-dimethyl-4H-benzo[d][1,3]dioxin-4-one (Compound 2)

To a solution of 5,7-dihydroxy-2,2-dimethyl-4H benzo[d][1,3]dioxin-4-one (6 g, 28.6 mmol) in tetrahydrofuran (100 mL) were added methanol (1.3 mL, 34.4 mmol) and triphenylphosphine (8.3 g, 31.42 mmol). After stirring for 5 min at 0° C., diethylazodicarboxylate (6.2 mL, 31.4 mmol) was added slowly to the reaction mixture and stirred for an additional 3 hrs at the same temperature. The remaining solvent was removed by evaporation under reduced pressure. The residue was purified by column chromatography (silica gel, EtOAc:n-hexane=1:9→1:4) to afford the title compound 5-hydroxy-7-methoxy-2,2-dimethyl-4H-benzo[d][1,3]dioxin-4-one as a white solid (5.2 g, yield 82%).

¹H NMR (400 MHz, CDCl₃) δ 10.45 (s, 1H), 6.15 (d, J=2.3 Hz, 1H), 6.00 (d, J=2.3 Hz, 1H), 3.82 (s, 3H), 1.73 (s, 6H).

### Step 3. 7-Methoxy-2,2-dimethyl-4-oxo-4H-benzo[d][1.3]dioxin-5-yltrifluoromethanesulfonate (Compound 3)

To a solution 5-hydroxy-7-methoxy-2,2-dimethyl-4H-benzo[d][1,3]dioxin-4-one (1.0 g, 4.46 mmol) in pyridine (10 mL) was slowly added trifluoromethanesulfonic anhydride (0.83 mL, 4.91 mmol) at 0° C., and the mixture was stirred for 5 hrs at the same temperature. The mixture was quenched with ice at 0° C., and extracted with ethyl acetate. The pooled organic layers were washed with 1 N HCl, dried over dry magnesium sulfate, and filtered. The filtrate was concentrated at a reduced pressure, and the residue was purified by column chromatography (silica gel, EtOAc:n-hexane=1:10→1:4) to afford the title compound 7-methoxy-2,2-dimethyl-4-oxo-4H-benzo[d][1.3]dioxin-5-yltrifluoromethane sulfonate as a white solid (1.5 g, yield 91%).

¹H NMR (300 MHz, CDCl₃) δ 6.53 (d, J=2.3 Hz 1H), 6.48 (d, J=2.3 Hz, 1H), 3.87 (s, 3H), 1.74 (s, 6H).

### Example 2

### Synthesis of (R)-tert-Butyl(pent-4-yn-2-yloxy)diphenylsilane (Compound 16a)

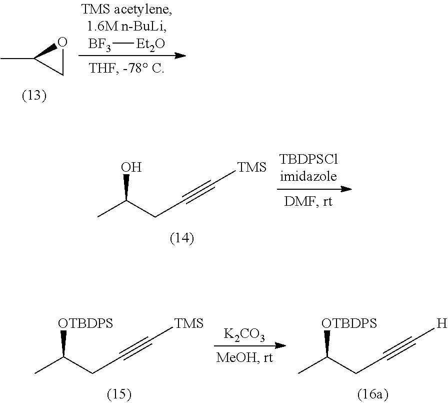

### Step 1. (R)-5-(Trimethylsilyl)pent-4-yn-2-ol

To a solution of ethynyltrimethylsilane (10.48 mL, 75.75 mmol) in dry tetrahydrofuran (60 mL) was added 1.6 M n-butyl lithioum (47 mL, 75.75 mmol) at −78° C. The reaction mixture was stirred for 0.5 hrs at the same temperature, slowly added with BF₃.Et₂O (8.65 mL, 68.87 mmol), and stirred again for 10 min. The reaction mixture was mixed with (R)-2-methyloxirane (4.0 g, 68.87 mmol)-78° C., and stirred for 2 hrs. After completion of the reaction, aqueous saturated ammonium chloride solution (3 mL) was added to the reaction mixture which was then extracted with ethyl acetate and water. The pooled organic layers were dried over anhydrous magnesium sulfate, and filtered. The filtrate was evaporated in a vacuum to dryness, and the residue was purified by column chromatography (silica gel, EtOAc:n-hexane=1:10) to afford the title compound (R)-5-(trimethylsilyl)pent-4-yn-2-ol as a colorless oil (7.6 g, yield 55%).

¹H NMR (400 MHz, CDCl₃) δ 3.97-3.93 (m, 1H), 2.51-2.32 (m, 2H), 1.90 (brs, 1H), 1.25 (d, J=6.1 Hz, 3H), 0.16 (s, 9H).

### Step 2. (R)-tert-Butyldiphenyl((5-(trimethylsilyl)pent-4-yn-2-yl)oxy)silane

To a solution of (R)-5-(trimethylsilyl)pent-4-yn-2-ol (2.1 g, 13.44 mmol) in N,N-dimethylformamide (20 mL) were added imidazole (1.37 g, 20.16 mmol) and tert-butylchlorodiphenylsilane (3.17 g, 13.44 mmol). The mixture was stirred at room temperature for 5 hrs. After completion of the reaction, the reaction mixture was extracted with ethyl acetate and water. The pooled organic layers were dried over anhydrous magnesium sulfate, and filtered. The filtrate was concentrated in a vacuum to dryness, and the residue was purified by column chromatography (silica gel, EtOAc:n-hexane=1:10) to afford the title compound (R)-tert-butyldiphenyl((5-(trimethylsilyl)pent-4-yn-2-yl)oxy)silane (4.8 g, yield 91%).

¹H NMR (400 MHz, CDCl₃) δ 7.59-7.55 (m, 4H), 7.31-7.22 (m, 6H), 3.89-3.85 (m, 1H), 2.31-2.17 (m, 2H), 1.04 (d, J=6.1 Hz, 3H), 0.93 (s, 9H), 0.01 (s, 9H).

### Step 3. (R)-tert-Butyl(pent-4-yn-2-yloxy)diphenylsilane

To a solution of (R)-tert-butyldiphenyl((5-(trimethylsilyl)pent-4-yn-2-yl)oxy)silane (8.1 g, 20.52 mmol) in methanol (120 mL) was added potassium carbonate (4.3 g, 30.78 mmol). This reaction mixture was stirred for 6 hrs at room temperature. After completion of the reaction, the reaction mixture was filtered through a pad of celite and concentrated by evaporation under reduced pressure. The reaction mixture was extracted with methylene chloride and water, and the pooled organic layers were dried over anhydrous magnesium sulfate, filtered, and concentrated under reduced pressure. The residue was purified by column chromatography (silica gel, EtOAc:n-hexane=1:10) to afford the title compound, (R)-tert-butyl(pent-4-yn-2-yloxy)diphenylsilane as a colorless oil (4.6 g, yield 70%).

¹H NMR (400 MHz, CDCl₃) δ 7.71-7.67 (m, 4H), 7.45-7.36 (m, 6H), 4.02-3.97 (m, 1H), 2.38-2.25 (m, 2H), 1.94 (t, J=2.6 Hz, 1H), 1.21 (d, J=6.0 Hz, 3H), 1.07 (s, 9H).

### Example 3

### Synthesis of (R)-tert-Butyldimethyl(pent-4-yn-2-yloxy)silane (Compound 16b)

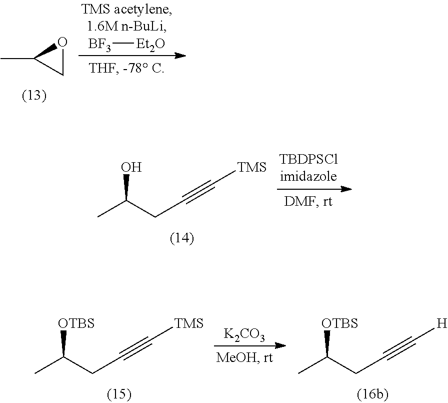

### Step 1. (R)-tert-Butyldimethyl(5-(trimethylsilyl)pent-4-yn-2-yloxy)silane

To a solution of (R)-5-(trimethylsilyl)pent-4-yn-2-ol (7.6 g, 29.4 mmol) in N,N-dimethylformamide (70 mL) were added imidazole (3.0 g, 44.1 mmol) and tert-butylchlorodimethylsilane (3.17 g, 13.44 mmol). The reaction mixture was stirred for 1 hr room temperature. After completion of the reaction, the reaction mixture was extracted with ethyl acetate and water. The pooled organic layers were dried over anhydrous magnesium sulfate, filtered, and concentrated by evaporation in a vacuum. The residue was purified by column chromatography (silica gel, EtOAc:n-hexane=1:10) to obtain the title compound, (R)-tert-butyldimethyl(5-(trimethylsilyl)pent-4-yn-2-yloxy) silane as a colorless oil (7.7 g, yield 97%).

¹H NMR (400 MHz, CDCl₃) δ 3.97-3.93 (m, 1H), 2.40-2.24 (m, 2H), 1.20 (d, J=6.0 Hz, 3H), 0.89 (s, 9H), 0.08 (d, J=4.2 Hz, 6H).

### Step 2. (R)-tert-Butyldimethyl(pent-4-yn-2-yloxy)silane

To a solution of (R)-tert-butyldimethyl(5-(trimethylsilyl)pent-4-yn-2-yloxy)silane (8.1 g, 20.52 mmol) in methanol (120 mL) was added potassium carbonate (4.6 g, 32.93 mmol), followed by stirring at room temperature for 4 hrs. After completion of the reaction, the reaction mixture was filtered through a pad of celite, and the solvent was removed by evaporation under reduced pressure. The concentrate was extracted with methylene and water, and the pooled organic layers were dried over anhydrous magnesium sulfate, filtered, and concentrated in a vacuum to dryness. The residue was purified by column chromatography (silica gel, EtOAc:n-hexane=1:10) to afford the title compound (R)-tert-butyldimethyl(pent-4-yn-2-yloxy)silane as a colorless oil (5.9 g, yield 94%).

¹H NMR (300 MHz, CDCl₃) δ 3.99-3.93 (m, 1H), 2.34-2.24 (m, 2H), 1.97 (t, J=2.7 Hz, 1H), 1.23 (d, J=6.0 Hz, 3H), 0.88 (s, 9H), 0.07 (d, J=2.1, 6H).

### Example 4

### Synthesis of (4S,5R)-Methyl 5-(3-(7-methoxy-2,2-dimethyl-4-oxo-4H-benzo[d][1,3]dioxin-5-yl)phenyl)-2,2-dimethyl-1,3-dioxorane-4-carboxylate (Compound 9)

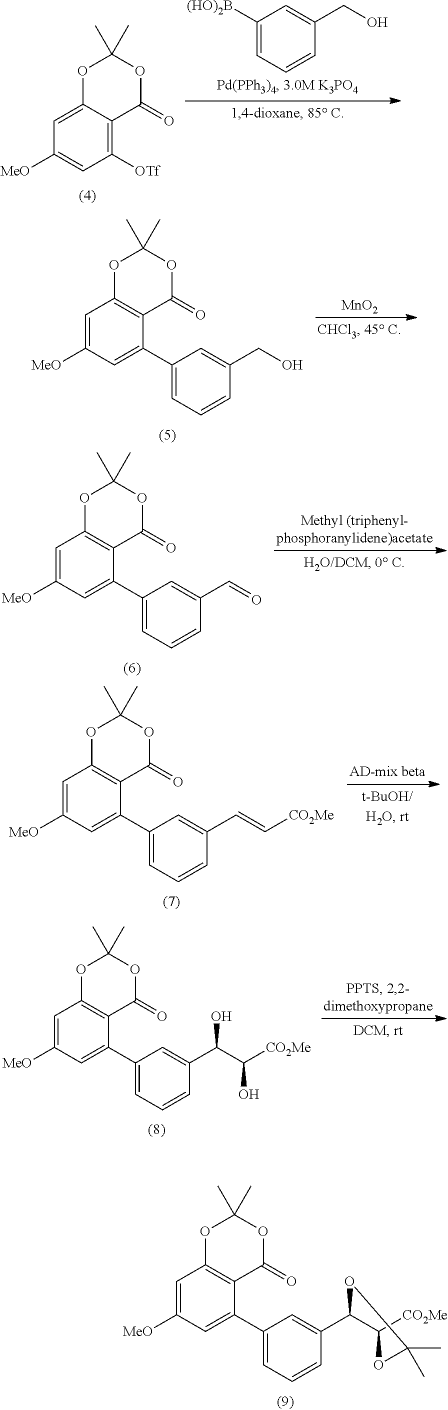

### Step 1. 5-(3-(Hydroxymethyl)phenyl)-7-methoxy-2,2-dimethyl-4H-benzo[d][1,3]dioxin-4-one (Compound 5)

To a solution of 7-methoxy-2,2-dimethyl-4-oxo-4H-benzo[d][1.3]dioxin-5-yl trifluoromethanesulfonate (9.6 g, 26.95 mmol), 3-(hydroxymethyl)phenylboronic acid (4.1 g, 26.95 mmol), and 3.0 M saturated potassium dihydrogen phosphate solution (26.95 mL) in 1,4-dioxane (100 mL) was added Pd(PPh₃)₄ (3.1 g, 2.70 mmol), and the reaction mixture was stirred at 85° C. for 1 hr. After completion of the reaction, the reaction mixture was filtered through a pad of celite, and extracted with ethyl acetate and water. The pooled organic layer was dried over anhydrous magnesium sulfate, filtered, and concentrated to dryness by evaporation in a vacuum. The residue was purified by column chromatography (silica gel, EtOAc:n-hexane=1:2→1:1) to afford the title compound 5-(3-(hydroxymethyl)phenyl)-7-methoxy-2,2-dimethyl-4H-benzo[d][1,3]dioxin-4-one as a white solid (7.0 g, yield 83%).

¹H NMR (400 MHz, DMSO-d₆) δ 7.32-7.31 (m, 2H), 7.26 (s, 1H), 7.19-7.16 (m, 1H), 6.68 (d, J=2.5 Hz, 1H), 6.54 (d, J=2.5 Hz, 1H), 5.22 (t, J=5.8 Hz, 1H), 4.53 (d, J=5.8 Hz, 2H), 3.86 (s, 3H), 1.74 (s, 6H).

### Step 2. 3-(7-Methoxy-2,2-dimethyl-4-oxo-4H-benzo[d][1,3]dioxin-5-yl)benzaldehyde (Compound 6)

To a solution of 5-(3-(hydroxymethyl)phenyl)-7-methoxy-2,2-dimethyl-4H-benzo[d][1,3]dioxin-4-one (7.1 g, 22.59 mmol) in chloroform (80 mL) was added activated manganese (IV) oxide (29.5 g, 338.9 mmol), followed by stirring at 45° C. for 6 hrs. After completion of the reaction, the reaction mixture was purified through a pad of celite, and concentrated by evaporation at a reduced pressure. The residue was purified by column chromatography (silica gel, EtOAc:n-hexane=1:2→1:1) to afford the title compound 3-(7-methoxy-2,2-dimethyl-4-oxo-4H-benzo[d][1,3]dioxin-5-yl)benzaldehyde as a white solid (5.8 g, yield 83%).

¹H NMR (400 MHz, DMSO-d₆) δ 10.05 (s, 1H), 7.91 (td, J=1.4 Hz, 1.4 Hz, 1H), 7.86 (t, J=1.5 Hz, 1H), 7.68 (td, J=1.6 Hz, 1.6 Hz, 1H), 7.61 (t, J=7.6 Hz, 1H), 6.74 (d, J=2.5 Hz, 1H), 6.64 (d, J=2.5 Hz, 1H), 3.88 (s, 3H), 1.76 (s, 6H).

### Step 3. (E)-methyl 3-(3-(7-methoxy-2,2-dimethyl-4-oxo-4H-benzo[d][1,3]dioxin-5-yl)phenyl)acrylate (Compound 7)

To a solution of 3-(7-methoxy-2,2-dimethyl-4-oxo-4H-benzo[d][1,3]dioxin-5-yl)benzaldehyde (1.0 g, 3.20 mmol) in a mixture of methylene chloride (1.7 mL) and water (15.7 mL) was added methyl(triphenylphosphoranylidene)acetate (1.28 g, 3.84 mmol) at 0° C., followed by stirring at the same temperature. After completion of the reaction, the reaction mixture was extracted with methylene chloride and water, and the pooled organic layer was dried over anhydrous magnesium sulfate, filtered, and concentrated to dryness by evaporation in a vacuum. The residue was purified by column chromatography (silica gel, EtOAc:DCM:n-hexane=0.5/1/8.5) to afford the title compound (E)-methyl 3-(3-(7-methoxy-2,2-dimethyl-4-oxo-4H-benzo[d][1,3]dioxin-5-yl)phenyl)acrylate as a white solid (1.01 g, yield 86%).

¹H NMR (400 MHz, CDCl₃) δ 7.71 (d, J=16 Hz, 1H), 7.53 (d, J=7.5 Hz, 1H), 7.45 (s, 1H), 7.40 (t, J=7.6 Hz, 1H), 7.33 (d, J=7.6 Hz, 1H), 6.52 (m, 1H), 6.46 (s, 1H), 6.44 (d, J=16 Hz, 1H), 3.86 (s, 3H), 3.79 (s, 3H), 1.77 (s, 6H).

### Step 4. (2S,3R)-Methyl 2,3-dihydroxy-3-(3-(7-methoxy-2,2-dimethyl-4-oxo-4H-benzo[d][1,3]dioxin-5-yl)phenyl)propanoate (Compound 8)

To a solution of 3-(3-(7-methoxy-2,2-dimethyl-4-oxo-4H-benzo[d][1,3]dioxin-5-yl)phenyl)acrylate (1 g, 2.71 mmol) in a mixture of tert-butanol (13 mL) and water (13 mL) were added methanesulfonamide (516 mg, 5.42 mmol) and AD-mix β (10 g) at ° C., followed by stirring at room temperature for 6 hrs. After completion of the reaction the reaction mixture was extracted with methylene chloride and water, and the pooled organic layer was dried over anhydrous magnesium sulfate, filtered, and concentrated to dryness by evaporation in a vacuum. The residue was purified by column chromatography (silica gel, DCM:MeOH=95:5) to afford the title compound (2S,3R)-methyl 2,3-dihydroxy-3-(3-(7-methoxy-2,2-dimethyl-4-oxo-4H-benzo[d][1,3]dioxin-5-yl)phenyl)propanoate as a white solid (916 mg, yield 84%).

¹H NMR (400 MHz, CDCl₃) δ 7.39-7.37 (m, 2H), 7.34 (s, 1H), 7.25-7.23 (m, 1H), 6.51 (d, J=2.5 Hz, 1H), 6.41 (d, J=2.5 Hz, 1H), 5.03 (dd, J=1.4 Hz, 3.4 Hz, 1H), 4.40 (dd, J=1.4 Hz, 3.1 Hz, 1H), 3.83 (s, 3H), 3.78 (s, 3H), 3.05 (d, J=6.1 Hz, 1H), 2.72 (d, J=6.9 Hz, 1H), 1.74 (s, 6H).

### Step 5. (4S,5R)-methyl 5-(3-(7-methoxy-2,2-dimethyl-4-oxo-4H-benzo[d][1,3]dioxin-5-yl)phenyl)-2,2-dimethyl-1,3-dioxorane-4-carboxylate (Compound 9)

To a solution of (2S,3R)-methyl 2,3-dihydroxy-3-(3-(7-methoxy-2,2-dimethyl-4-oxo-4H-benzo[d][1,3]dioxin-5-yl)phenyl)propanoate (3.03 g, 7.53 mmol) in methylene chloride (30 mL) were added pyridium p-toluenesulfonate (568 mg, 2.26 mmol) and 2,2-dimethoxypropane (13.9 mL, 112.95 mmol), followed by stirring at room temperature for 16 hrs. After completion of the reaction the reaction mixture was extracted with methylene chloride and water, and the pooled organic layer was dried over anhydrous magnesium sulfate, filtered, and concentrated to dryness by evaporation in a vacuum. The residue was purified by column chromatography (silica gel, EtOAc:n-hexane=1:2) to afford the title compound (4S,5R)-methyl 5-(3-(7-methoxy-2,2-dimethyl-4-oxo-4H-benzo[d][1,3]dioxin-5-yl)phenyl)-2,2-dimethyl-1,3-dioxorane-4-carboxylate as a colorless oil (2.5 g, yield 76%).

¹H NMR (400 MHz, CDCl₃) δ 7.44-7.40 (m, 2H), 7.38 (s, 1H), 7.31-7.28 (m, 1H), 6.53 (d, J=2.5 Hz, 1H), 6.45 (d, J=2.5 Hz, 1H), 5.22 (d, J=7.5 Hz, 1H), 4.43 (d, J=7.5 Hz, 1H), 3.86 (s, 3H), 3.79 (s, 3H), 1.77 (s, 6H), 1.58 (s, 3H), 1.55 (s, 3H).

### Example 5

### Synthesis of Methyl 3′-((4R,5R)-5-(hydroxymethyl)-2,2-dimethyl-1,3-dioxoran-4-yl)-5-methoxy-3-(methoxymethoxy)-[1,1′-biphenyl]-2-carboxylate (Compound 12)

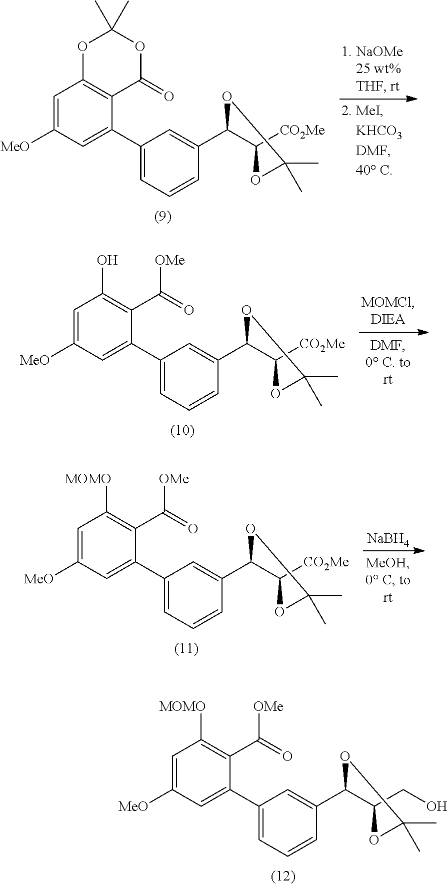

### Step 1. (4S,5R)-methyl 5-(3′-hydroxy-5′-methoxy-2′-(methoxycarbonyl)-[1,1′-biphenyl]-3-yl)-2,2-dimethyl-1,3-dioxorane-4-carboxylate (Compound 10)

To a solution of (4S,5R)-methyl 5-(3-(7-methoxy-2,2-dimethyl-4-oxo-4H-benzo[d][1,3]dioxin-5-yl)phenyl)-2,2-dimethyl-1,3-dioxorane-4-carboxylate (460 mg, 1.04 mmol) in tetrahydrofuran (5.5 mL) was added a 25 wt % sodium methoxide solution in methanol (1.16 mL) at 0° C., followed by stirring at room temperature for 0.5 hrs. After completion of the reaction, the reaction mixture was neutralized with a saturated aqueous ammonium chloride solution. Then, the reaction mixture was extracted with methylene chloride and water, and the pooled organic layer was dried over anhydrous magnesium sulfate, filtered, and concentrated to dryness by evaporation in a vacuum. The residue (420 mg) was used for the next reaction without further purification. The residue (420 mg) was dissolved, together with potassium hydrogen carbonate (126 mg, 1.25 mmol) and methyl iodide (0.11 mL, 1.56 mmol), in N,N-dimethylformamide (2.2 mL) at 40° C. for 6 hrs with stirring. After completion of the reaction, the reaction mixture was extracted with ethyl acetate and water, and the pooled organic layers were dried over magnesium sulfate, filtered, and concentrated to dryness by evaporation in a vacuum. The concentrate was purified by column chromatography (silica gel, EtOAc:n-hexane=1:4) to afford the title compound (4S,5R)-methyl 5-(3′-hydroxy-5′-methoxy-2′-(methoxycarbonyl)-[1,1′-biphenyl]-3-yl)-2,2-dimethyl-1,3-dioxorane-4-carboxylate as a colorless oil (368 mg, yield 85%).

¹H NMR (400 MHz, CDCl₃) δ 11.28 (s, 1H), 7.35-7.30 (m, 2H), 7.17-7.14 (m, 2H), 6.45 (d, J=2.6 Hz, 1H), 6.29 (d, J=2.6 Hz, 1H), 5.13 (d, J=7.6 Hz, 1H), 4.31 (d, J=7.6 Hz, 1H), 3.79 (s, 3H), 3.75 (s, 3H), 3.40 (s, 3H), 1.56 (s, 3H), 1.52 (s, 3H).

### Step 2. (4S,5R)-methyl 5-(5′-methoxy-2′-(methoxycarbonyl)-3′-(methoxymethoxy)-[1,1′-biphenyl]-3-yl)-2,2-dimethyl-1,3-dioxorane-4-carboxylate (Compound 11)

To a solution of (4S,5R)-methyl 5-(3′-hydroxy-5′-methoxy-2′-(methoxycarbonyl)-[1,1′-biphenyl]-3-yl)-2,2-dimethyl-1,3-dioxorane-4-carboxylate (2.35 g, 5.65 mmol) and N,N-diisopropylethylamine (4.9 mL, 28.3 mmol) in N,N-dimethylformamide (20 mL) was slowly added methoxymethyl chloride (MOMC1; 1.0 mL, 11.3 mmol) at 0° C., followed by stirring at room temperature for 2 hrs. After completion of the reaction, the reaction mixture was extracted with ethyl acetate and water, and the pooled organic layers were dried over magnesium sulfate, dried over magnesium sulfate, filtered, and concentrated to dryness by evaporation in a vacuum. The residue was purified by column chromatography (silica gel, EtOAc:n-hexane=1:4→:2) to afford the title compound (4S,5R)-methyl 5-(5′-methoxy-2′-(methoxycarbonyl)-3′-(methoxymethoxy)-[1,1′-biphenyl]-3-yl)-2,2-dimethyl-1,3-dioxorane-4-carboxylate as a colorless oil (2.4 g, yield 91%).

¹H NMR (400 MHz, CDCl₃) δ 7.43 (s, 1H), 7.37-7.36 (m, 2H), 7.34-7.31 (m, 1H), 6.72 (d, J=2.2 Hz, 1H), 6.53 (d, J=2.3 Hz, 1H), 5.19 (s, 2H), 5.15 (d, J=7.6 Hz, 1H), 4.34 (d, J=7.6 Hz, 1H), 3.81 (s, 3H), 3.77 (s, 3H), 3.55 (s, 3H), 3.47 (s, 3H), 1.59 (s, 3H), 1.52 (s, 3H).

### Step 3. Methyl 3′-((4R,5R)-5-(hydroxymethyl)-2,2-dimethyl-1,3-dioxoran-4-yl)-5-methoxy-3-(methoxymethoxy)-[1,1′-biphenyl]-2-carboxylate (Compound 12)

To a solution of (4S,5R)-methyl 5-(5′-methoxy-2′-(methoxycarbonyl)-3′-(methoxymethoxy)-[1,1′-biphenyl]-3-yl)-2,2-dimethyl-1,3-dioxorane-4-carboxylate (2.55 g, 5.54 mmol) in methanol (25 mL) was slowly added sodium borohydride (838 mg, 22.2 mmol) at 0° C., followed by stirring at room temperature for 4 hrs. After completion of the reaction, the reaction mixture was neutralized with an aqueous saturated ammonium chloride solution, and extracted with methylene chloride and water, and the pooled organic layers were dried over anhydrous magnesium sulfate, filtered and concentrated to dryness by evaporation in a vacuum. The residue was purified by column chromatography (silica gel, EtOAc:n-hexane=1:1) to afford the title compound methyl 3′-((4R,5R)-5-(hydroxymethyl)-2,2-dimethyl-1,3-dioxoran-4-yl)-5-methoxy-3-(methoxymethoxy)-[1,1′-biphenyl]-2-carboxylate as a colorless oil (2.1 g, yield 82%).

¹H NMR (400 MHz, CDCl₃) δ 7.41 (s, 1H), 7.38 (d, J=6.3 Hz, 2H), 7.36-7.32 (m, 1H), 6.74 (d, J=2.2 Hz, 1H), 6.54 (d, J=2.2 Hz, 1H), 5.21 (s, 2H), 4.91 (d, J=8.4 Hz, 1H), 3.90-3.85 (m, 2H), 3.83 (s, 3H), 3.69-3.63 (m, 1H), 3.58 (s, 3H), 3.49 (s, 3H), 1.94-1.91 (m, 1H), 1.57 (s, 3H), 1.52 (s, 3H).

### Example 6

### Synthesis of Methyl 3′-((4R,5R)-5-((5R,Z)-5-hydroxy-1-((4-methoxybenzyl)oxy)hex-2-en-1-171)-2,2-dimethyl-1,3-dioxoran-4-yl)-5-methoxy-3-(methoxymethoxy)-[1,1′-biphenyl]-2-carboxylate (Compound 20)

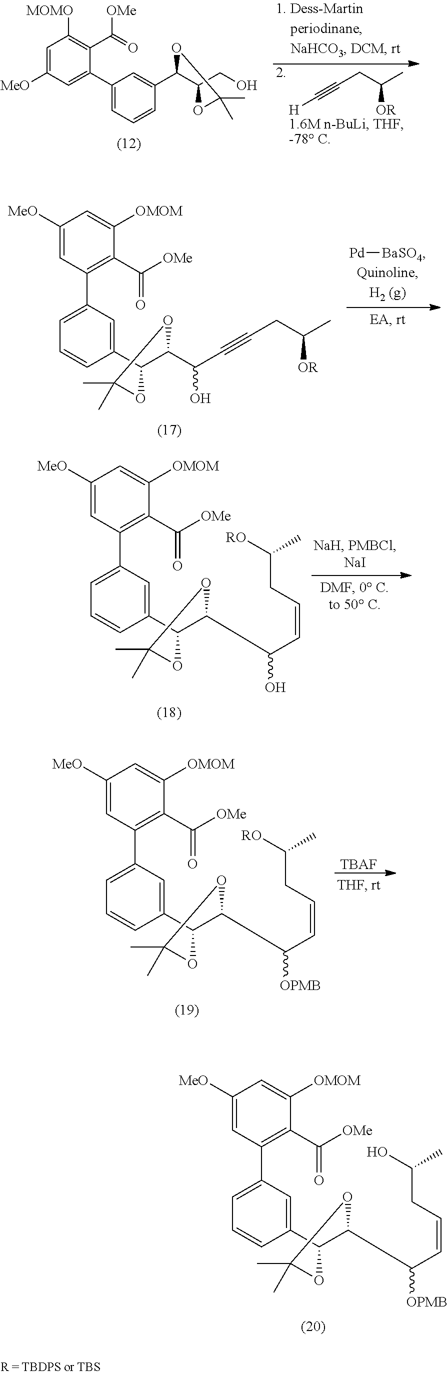

### Step 1-1. Methyl 3′-(4R,5R)-5-((5R)-5-(tert-butyldiphenylsilyl)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate (Compound 17-1)

To a solution of methyl 3′-((4R,5R)-5-(hydroxymethyl)-2,2-dimethyl-1,3-dioxoran-4-yl)-5-methoxy-3-(methoxymethoxy)-[1,1′-biphenyl]-2-carboxylate (500 mg, 1.16 mmol) in methylene chloride (5 mL) were sodium hydrogen carbonate (292 mg, 3.48 mmol) and Dess-Martin periodianane (734 mg, 1.73 mmol), followed by stirring for 3 hrs at room temperature. After completion of the reaction, the reaction mixture was diluted in methylene chloride and extracted with saturated sodium hydrogen carbonate and saturated Na₂S₂O₃ solutions. The pooled organic layers were dried over anhydrous magnesium sulfate, filtered, and concentrated in a vacuum by evaporation. The residue (422 mg) was used in the next step without further purification. Separately, (R)-tert-butyl(pent-4-yn-2-yloxy)diphenylsilane (948 mg, 2.94 mmol) was dissolved in dry tetrahydrofuran (4 mL), and cooled to −78° C., after which 1.6 M n-butyllithium (1.78 mL, 2.84 mmol) was slowly dropwise added to this solution, and stirred for 1.5 hrs at the same time. To this solution was slowly dropwise added a solution of the residue (422 mg, 0.98 mmol) in dry tetrahydrofuran (4 mL) at −78° C., followed by stirring for 2 hrs. After completion of the reaction, the reaction mixture was added with an aqueous saturated ammonium chloride solution (3 mL), and extracted with ethyl acetate and water. The pooled organic layers were dried over anhydrous magnesium sulfate, filtered and concentrated to dryness by evaporation in a vacuum. The residue was purified by column chromatography (silica gel, EtOAc:n-hexane=1:4→1:2) to afford the title compound methyl 3′-(4R,5R)-5-((5R)-5-(tert-butyldiphenylsilyl)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate as a colorless oil (369 mg, two steps, yield 47%).

¹H NMR (400 MHz, CDCl₃) δ 7.66-7.63 (m, 4H), 7.43-7.32 (m, 10H), 6.75 (d, J=2.2 Hz, 1H), 6.53 (d, J=2.2 Hz, 1H), 5.22 (s, 2H), 4.99-4.93 (m, 1H), 4.44-4.32 (m, 1H), 3.98-3.88 (m, 2H), 3.83-3.82 (m, 3H), 3.57-3.56 (m, 3H), 3.50 (s, 3H), 2.47-2.30 (m, 1H), 2.25-2.18 (m, 1H), 1.56 (m, 3H), 1.50 (d, J=7.1 Hz, 3H), 1.15-1.09 (m, 3H), 1.03 (d, J=6.2 Hz, 9H).

### Step 1-2. methyl 3′-(4R,5R)-5-((5R)-5-((tert-butyldimethylsilyl)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate (Compound 17-2)

To a solution of methyl 3′-((4R,5R)-5-(hydroxymethyl)-2,2-dimethyl-1,3-dioxoran-4-yl)-5-methoxy-3-(methoxymethoxy)-[1,1′-biphenyl]-2-carboxylate (500 mg, 1.16 mmol) in methylene chloride (5 mL) were added sodium hydrogen carbonate (292 mg, 3.48 mmol) and Dess-Martin periodianane (734 mg, 1.73 mmol), followed by stirring at room temperature for 3 hrs. After completion of the reaction, the reaction mixture was diluted in methylene chloride and extracted with saturated sodium hydrogen carbonate and saturated Na₂S₂O₃ solutions. The pooled organic layers were dried over anhydrous magnesium sulfate, filtered, and concentrated in a vacuum by evaporation. The residue (422 mg) was used in the next step without further purification. Separately, (R)-tert-butyldimethyl(pent-4-yn-2-yloxy)silane (593 mg, 2.94 mmol) was dissolved in dry tetrahydrofuran (4 mL), and cooled to −78° C., after which 1.6 M n-butyllithium (1.78 mL, 2.84 mmol) was slowly dropwise added to this solution, and stirred for 1.5 hrs at the same time. To this solution was slowly dropwise added a solution of the residue (422 mg, 0.98 mmol) in dry tetrahydrofuran (4 mL) at −78° C., followed by stirring for 4 hrs. After completion of the reaction, the reaction mixture was mixed with an aqueous saturated ammonium chloride solution (3 mL) and extracted with ethyl acetate and water. The pooled organic layers were dried over anhydrous magnesium sulfate, filtered and concentrated to dryness by evaporation in a vacuum. The residue was purified by column chromatography (silica gel, EtOAc:n-hexane=1:4→1:2) to afford the title compound methyl 3′-(4R,5R)-5-((5R)-5-((tert-butyldimethylsilyl)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate as a colorless oil (328 mg, two step, yield 45%).

¹H NMR (400 MHz, CDCl₃) δ 7.45 (s, 1H), 7.40-7.31 (s, 3H), 6.73 (d, J=2.2 Hz, 1H), 6.54 (d, J=2.2 Hz, 1H), 5.20 (s, 2H), 5.04-4.95 (m, 1H), 4.51-4.40 (m, 1H), 4.03-3.92 (m, 1H), 3.90-3.84 (m, 1H), 3.82 (s, 3H), 3.58 (s, 3H), 3.48 (s, 3H), 2.34-2.56 (s, 2H), 2.20-2.13 (m, 1H), 1.57 (d, J=7.1 Hz, 3H), 1.52 (m, 3H), 1.56-1.11 (m, 3H), 0.85-0.84 (m, 9H), 0.02-0.01 (m, 6H).

### Step 2-1. methyl 3′-((4R,5R)-5-((5R,Z)-5-((tert-butyldiphenylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate (Compound 18-1)

To a solution methyl 3′-(4R,5R)-5-((5R)-5-(tert-butyldiphenylsilyl)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate (510 mg, 0.68 mmol) in ethyl acetate (3.0 mL) were added quinoline (0.18 mL) and Pd—BaSO₄ (106 mg), followed by stirring at room temperature for 8 hrs under a hydrogen atmosphere. After completion of the reaction, the reaction mixture was filtered through a pad of celite, and extracted with water and ethyl acetate. The pooled organic layers were dried over anhydrous magnesium sulfate, filtered and concentrated to dryness by evaporation in a vacuum. The residue was purified by column chromatography (silica gel, EtOAc:n-hexane=1:4→1:2) to afford the title compound methyl 3′-((4R,5R)-5-((5R,Z)-5-((tert-butyldiphenylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate as a colorless oil (416 mg, yield 80%).

¹H NMR (400 MHz, CDCl₃) δ 7.65-7.63 (m, 4H), 7.42-7.30 (m, 10H), 6.74 (d, J=2.2 Hz, 1H), 6.52 (t, J=2.0 Hz, 1H), 5.59-5.54 (m, 1H), 5.50-5.44 (m, 1H), 5.21 (s, 2H), 4.88 (d, J=8.3 Hz, 1H), 4.47-4.25 (m, 1H), 3.93-3.83 (m, 2H), 3.82-3.81 (m, 3H), 3.55-3.53 (m, 3H), 3.49 (s, 3H), 2.34-02.12 (m, 2H), 1.55 (d, J=6.9 Hz, 3H), 1.50 (m, 3H), 1.03-1.02 (m, 9H), 1.00-0.99 (m, 3H).

### Step 2-2. Methyl 3′-((4R,5R)-5-((5R,Z)-5-((tert-butyldimethylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate (Compound 18-2)

To a solution of methyl 3′-(4R,5R)-5-((5R)-5-((tert-butyldimethylsilyl)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate (77 mg, 0.12 mmol) in ethyl acetate and quinoline (0.8 mL) were added quinoline (33 μl) and Pd—BaSO₄ (7.7 mg), followed by stirring at room temperature for 8 hrs under a hydrogen atmosphere. After completion of the reaction, the reaction mixture was filtered through a pad of celite, and extracted with water and ethyl acetate. The pooled organic layers were dried over anhydrous magnesium sulfate, filtered and concentrated to dryness by evaporation in a vacuum. The residue was purified by column chromatography (silica gel, EtOAc:n-hexane=1:4→1:2) to afford the title compound methyl 3′-((4R,5R)-5-((5R,Z)-5-((tert-butyldimethylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate as a colorless oil (62 mg, yield 82%).

¹H NMR (400 MHz, CDCl₃) δ 7.40 (s, 1H), 7.43-7.28 (m, 3H), 6.72 (d, J=2.0 Hz, 1H), 6.52 (d, J=2.0 Hz, 1H), 5.65-5.46 (m, 1H), 5.20 (s, 2H), 4.93-4.89 (m, 1H), 4.56-4.40 (m, 1H), 4.00 (dd, J=1.8 Hz, 4.0 Hz, 1H), 3.87-3.84 (m, 1H), 3.81 (s, 3H), 3.57-3.55 (m, 3H), 3.48 (s, 3H), 2.28-2.22 (m, 2H), 1.54-1.49 (m, 6H), 1.07-1.06 (m, 3H), 0.85-0.84 (m, 9H), 0.02-0.01 (m, 6H).

### Step 3-1. Methyl 3′-((4R,5R)-5-((5R,Z)-5-((tert-butyldiphenylsilyl)oxy)-1-((4-methoxybenzyl)oxy)hex-2-en-1-yl)-2,2-dimethyl-1,4-dioxoran-4-yl)-5-methoxy-3-(methoxymethoxy)-[1,1′-biphenyl]-2-carboxylate (Compound 19-1)

To a solution of methyl 3′-((4R,5R)-5-((5R,Z)-5-((tert-butyldiphenylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate (992 mg, 1.31 mmol) in N,N-dimethylformamide (5 mL) was added 60% sodium hydride (142 mg, 3.55 mmol) at 0° C., followed by stirring for 0.5 hrs. The resulting mixture was further mixed with sodium iodide (589 mg, 3.93 mmol) and p-methoxybenzyl chloride (0.48 mL, 3.55 mmol) at 50° C. for 6 hrs, with stirring. After completion of the reaction, the reaction mixture was quenched with ice water, and extracted with ethyl acetate and water. The pooled organic layers were dried over anhydrous magnesium sulfate, filtered and concentrated to dryness by evaporation in a vacuum. The residue was purified by column chromatography (silica gel, EtOAc:n-hexane=1:4→1:2) to afford the title compound methyl 3′-((4R,5R)-5-((5R,Z)-5-((tert-butyldiphenylsilyl)oxy)-1-((4-methoxybenzyl)oxy)hex-2-en-1-yl)-2,2-dimethyl-1,4-dioxoran-4-yl)-5-methoxy-3-(methoxymethoxy)-[1,1′-biphenyl]-2-carboxylate as a colorless oil (436 mg, yield 38%).

MS m/z calcd for C₅₂H₆₂O₁₀Si [M+Na]⁺898.13. found. 897.72.

### Step 3-2. Methyl 3′-((4R,5R)-5-((5R,Z)-5-((tert-butyldimethylsilyl)oxy)-1-((4-methoxy benzyl)oxy)hex-2-en-2-yl)-2,2-dimethyl-1,3-dioxoran-4-yl)-5-methoxy-3-(methoxymethoxy)-[1,1′-biphenyl]-2-carboxylate (Compound 19-2)

To a solution of methyl 3′-((4R,5R)-5-((5R,Z)-5-((tert-butyldimethylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate (65 mg, 0.10 mmol) in N,N-dimethylformamide (0.35 mL) was added 60% sodium hydride (12 mg, 0.30 mmol), followed by stirring for 0.5 hrs. The reaction mixture was further mixed with sodium iodide (45 mg, 0.30 mmol) and p-methoxybenzyl chloride (37 μl, 0.27 mmol) at 50° C. for 2 hrs, with stirring. After completion of the reaction, the reaction mixture was quenched with ice water, and extracted with ethyl acetate and water. The pooled organic layers were dried over anhydrous magnesium sulfate, filtered and concentrated to dryness by evaporation in a vacuum. The residue was purified by column chromatography (silica gel, EtOAc:n-hexane=1:4→1:2) to afford the title compound methyl 3′-((4R,5R)-5-((5R,Z)-5-((tert-butyldimethylsilyl)oxy)-1-((4-methoxybenzyl)oxy)hex-2-en-2-yl)-2,2-dimethyl-1,3-dioxoran-4-yl)-5-methoxy-3-(methoxymethoxy)-[1,1′-biphenyl]-2-carboxylate as a colorless oil (54 mg, yield 70%).

¹H NMR (400 MHz, CDCl₃) δ 7.45 (s, 1H), 7.40-7.30 (m, 3H), 7.14 (d, J=8.6 Hz, 2H), 6.84-6.76 (m, 2H), 6.73 (d, J=2.2 Hz, 1H), 6.51-6.49 (m, 1H), 5.83-5.73 (m, 1H), 5.52-5.21 (m, 1H), 5.21 (s, 2H), 4.95-4.87 (m, 1H), 4.62-4.50 (m, 1H), 4.30-4.25 (m, 1H), 4.09-4.04 (m, 1H), 3.90-3.82 (m, 2H), 3.81-3.80 (m, 3H), 3.76-3.75 (m, 3H), 3.54-3.53 (m, 3H), 3.49 (s, 3H), 2.21-2.16 (m, 2H), 1.53-1.45 (m, 6H), 1.10-1.04 (m, 3H), 0.87-0.85 (m, 9H), 0.03-0.01 (m, 6H).

### Step 4. Methyl 3′-((4R,5R)-5-((5R,Z)-5-hydroxy-1-((4-methoxybenzyl)oxy)hex-2-en-1-yl)-2,2-dimethyl-1,3-dioxoran-4-yl)-5-methoxy-3-(methoxymethoxy)-[1,1′-biphenyl]-2-carboxylate (Compound 20)

To a solution of methyl 3′-((4R,5R)-5-((5R,Z)-5-((tert-butyl [symbol] phenylsilyl)oxy)-1-((4-methoxy benzyl)oxy)hex-2-en-1-yl)-2,2-dimethyl-1,4-dioxoran-4-yl)-5-methoxy-3-(methoxymethoxy)-[1,1′-biphenyl]-2-carboxylate (300 mg, 0.34 mmol) in tetrahydrofuran (1.5 mL) was added 1.0 M tetrabutylammonium fluoride (3.43 mL, 3.43 mmol), followed by stirring at room temperature for 18 hrs. After completion of the reaction, the reaction mixture was extracted with ethyl acetate and water, the pooled organic layers were dried over anhydrous magnesium sulfate, filtered and concentrated to dryness by evaporation in a vacuum. The residue was purified by column chromatography (silica gel, EtOAc:n-hexane=1:2) to afford the title compound methyl 3′-((4R,5R)-5-((5R,Z)-5-hydroxy-1-((4-methoxybenzyl)oxy)hex-2-en-1-yl)-2,2-dimethyl-1,3-dioxoran-4-yl)-5-methoxy-3-(methoxymethoxy)-[1,1′-biphenyl]-2-carboxylate as a colorless oil (210 mg, yield 97%).

MS m/z calcd for C₃₆H₄₄O₁₀ [M+Na]⁺659.73. found. 659.30.

### Example 7

### Synthesis of (7S,12S,13R,Z)-4,12,13-Trihydroxy-2-methoxy-7-methyl-7,8,13,14-tetrahydro-5H-dibenzo[c,e][1]oxacyclotetradecen-5,11(12H)-dione (Compound 24)

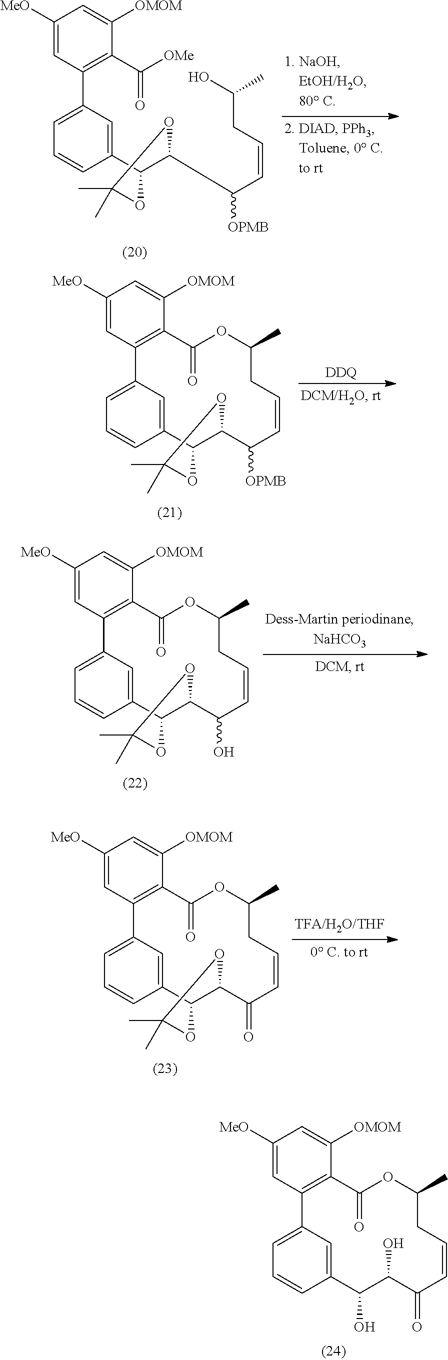

### Step 1. (3aR,8S,19aR, Z)-13-Methoxy-4-((4-methoxybenzyl)oxy)-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one (Compound 21)

To a solution of methyl 3′-((4R,5R)-5-((5R,Z)-5-hydroxy-1-((4-methoxybenzyl)oxy)hex-2-en-1-yl)-2,2-dimethyl-1,3-dioxoran-4-yl)-5-methoxy-3-(methoxymethoxy)-[1,1′-biphenyl]-2-M carboxylate (230 mg, 0.36 mmol) in ethanol (3.2 mL) were added sodium hydroxide (722 mg, 18.05 mmol) and (3.2 mL), followed by stirring at 80° C. for 8 hrs under reflux. After completion of the reaction, the reaction mixture was neutralized to pH 6.0 with 6 N HCl, and extracted with methylene chloride and water. The pooled organic layer was dried over anhydrous magnesium sulfate, filtered, and concentrated by evaporation in a vacuum. The residue (220 mg) was used in the next step without further purification. The residue (220 mg, 0.35 mol) was dissolved, together with triphenylphosphine (185 mg, 0.71 mmol), in dry toluene (1.5 mL), and diisopropylazodicarboxylate (0.14 mL, 0.71 mmol) was slowly added at 0° C. to the solution which was then stirred at room temperature for 0.5 hrs. After completion of the reaction, the reaction mixture was quenched with a saturated ammonium chloride solution (3 mL), and extracted with ethyl acetate and water. The pooled organic layers were dried over anhydrous magnesium sulfate, filtered and concentrated to dryness by evaporation in a vacuum. The residue was purified by column chromatography (silica gel, EtOAc:n-hexane=1:4→1:2) to afford the title compound (3aR,8S,19aR, Z)-13-methoxy-4-((4-methoxybenzyl)oxy)-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one (160 mg) as a yellow solid.

MS m/z calcd for C₃₅H₄₀O₉ [M+Na]⁺ 627.69. found. 626.81.

### Step 2. (3aR,8S,19aR,Z)-4-hydroxy-13-methoxy-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one (Compound 22)

To a solution of (3aS,8S,19aR,Z)-13-methoxy-4-((4-methoxybenzyl)oxy)-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one (160 mg, 0.26 mmol) in methylene chloride (1.5 mL) were added water (0.1 mL) and 2,3-dichloro-5,6-dicyano-1,4-benzoquinone (66 mg, 0.29 mmol), followed by stirring at room temperature for 1 hr. After completion of the reaction, the reaction mixture was extracted with methylene chloride and water, and the pooled organic layers were dried over anhydrous magnesium sulfate, filtered and concentrated to dryness by evaporation in a vacuum. The residue was purified by column chromatography (silica gel, EtOAc:n-hexane=1:4-1:2) to afford the title compound (3aR,8S,19aR,Z)-4-hydroxy-13-methoxy-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one as a white solid (47 mg, three steps yield 32%).

MS m/z calcd for C₂₇H₃₂O₈ [M+Na]⁺ 507.54. found. 506.89.

### Step 3. (3aS,8S,19aR,Z)-13-methoxy-11-(methoxymethoxy)-2,2,8-trimethyl-7,8,19,19a-tetrahydro-3aH-dibenzo[c,e][1.3]dioxolo[4,5-h][1]oxacyclotetradecen-4,10-dione (Compound 23)

To a solution of (3aR,8S,19aR,Z)-4-hydroxy-13-methoxy-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one (47 mg, 0.097 mmol) in methylene chloride (0.6 mL) were added sodium hydrogen carbonate (24.4 mg, 0.291 mmol) and Dess-Martin periodianane (61.7 mg, 0.145 mmol), followed by stirring at room temperature for 1 hr. After completion of the reaction, the reaction mixture was diluted in methylene chloride, and extracted with a saturated sodium hydrogen carbonate solution and a saturated Na₂S₂O₃ solution. The pooled organic layers were dried over anhydrous magnesium sulfate, filtered, and concentrated to dryness by evaporation in a vacuum. The residue was purified by column chromatography (silica gel, EtOAc:n-hexane=1:2→1:1) to afford the title compound (3aS,8S,19aR,Z)-13-methoxy-11-(methoxymethoxy)-2,2,8-trimethyl-7,8,19,19a-tetrahydro-3aH-dibenzo[c,e][1.3]dioxolo[4,5-h][1]oxacyclotetradecen-4,10-dione as a white solid (26 mg, yield 55%).

¹H NMR (400 MHz, CDCl₃) δ 7.59 (d, J=7.7 Hz, 1H), 7.51 (t, J=10.1 Hz, 1H), 7.42 (d, J=7.5 Hz, 1H), 7.06 (s, 1H), 6.71 (d, J=2.1 Hz, 1H), 6.38 (d, J=2.2 Hz, 1H), 6.30 (td, J=2.9 Hz, 11.0 Hz, 1H), 5.78 (dd, J=1.4 Hz, 5.7 Hz, 1H), 5.34-5.30 (m, 1H), 5.22 (d, J=6.8 Hz, 1H), 5.15 (d, J=6.8 Hz, 1H), 4.58 (q, J=7.5 Hz, 2H), 3.80 (s, 3H), 3.48 (s, 3H), 3.21-3.11 (m, 1H), 2.54-2.49 (m, 1H), 1.62 (d, J=7.8 Hz, 6H), 1.42 (d, J=6.5 Hz, 3H) m/z calcd for C₂₇H₃₀O₈ [M+Na]⁺ 505.52. found 504.87.

### Step 4. (7S,12S,13R,Z)-4,12,13-trihydroxy-2-methoxy-7-methyl-7,8,13,14-tetrahydro-5H-dibenzo[c,e][1]oxacyclotetradecen-5,11(12H)-dione (Compound 24)

To a solution of (3aS,8S,19aR,Z)-13-methoxy-11-(methoxymethoxy)-2,2,8-trimethyl-7,8,19,19a-tetrahydro-3aH-dibenzo[c,e][1.3]dioxolo[4,5-h][1]oxacyclotetradecen-4,10-dione (14.5 mg, 0.03 mmol) in tetrahydrofuran (0.2 mL) were added water (0.1 mL) and trifluoroacetic acid (0.2 mL) at 0° C., followed by stirring at room temperature for 2 hrs. After completion of the reaction, the reaction mixture was neutralized with a saturated sodium hydrogen carbonate solution, and extracted with methylene chloride and water. The pooled organic layers were dried over anhydrous magnesium sulfate, filtered, and concentrated to dryness by evaporation in a vacuum. The residue was purified by column chromatography (silica gel, EtOAc:DCM=1:3) to afford the title compound (7S,12S,13R,Z)-4,12,13-trihydroxy-2-methoxy-7-methyl-7,8,13,14-tetrahydro-5H-benzo[c,e][1]oxacyclotetradecen-5,11(12H)-dione as a white solid (4.3 mg, yield 36%).

TLC Rf 0.30 (1/3, EtOAc/DCM). ¹H NMR (400 MHz, CDCl₃) δ 10.04 (s, 1H), 7.77 (d, J=7.7 Hz, 1H), 7.53 (s, 1H), 7.52-7.50 (m, 1H), 7.11 (d, J=7.8 Hz), 6.52 (d, J=2.2 Hz, 1H), 6.30 (d, J=2.1 Hz, 1H), 5.90-5.82 (m, 1H), 5.34-5.32 (m, 1H), 5.28-5.27 (m, 1H), 5.25-5.23 (m, 1H), 4.39 (d, J=8.4 Hz, 1H), 3.84 (s, 3H), 3.81-3.90 (m, 1H), 1.82-1.80 (m, 2H), 1.44 (d, J=6.4 Hz, 3H). MS m/z calcd for C₂₂H₂₂O₇ [M+Na]⁺ 421.41. found 420.92.

The novel compound of Chemical Formula F according to the present invention may be formulated into various forms according to purpose. Several formulations containing the compound of Chemical Formula F as an active ingredient are illustrated, below, but are not set forth to limit the present invention.

### FORMULATION EXAMPLES

### Formulation Example 1

### Tablet (Direct Compression)

After being sieved, 5.0 mg of the active ingredient was blended with 14.1 mg of lactose, 0.8 mg of crospovidone USNF, and 0.1 mg of magnesium stearate, and compressed into a tablet.

### Formulation Example 2

### Tablet (Wet Granulation)

After being sieved, 5.0 mg of the active ingredient was blended with 16.0 mg of lactose and 4.0 mg of starch. A solution of 0.3 mg of polysolvate 80 in pure water was added in an adequate amount to the blend which was then finely powered. The fine powders were dried, sieved, and admixed with 2.7 mg of colloidal silicon dioxide and 2.0 mg of magnesium stearate, and compressed into a tablet.

### Formulation Example 3

### Powder and Capsule

After being sieved, 5.0 mg of the active ingredient was blended with 14.8 mg of lactose, 10.0 mg of polyvinylpyrrolidone, and 0.2 mg of magnesium stearate. The resulting blend was loaded into a hard No. 5 gelatin capsule.

### Formulation Example 4

### Injection

An injection was prepared by dissolving 100 mg of the active ingredient, together with 180 mg of mannitol, and 26 mg of Na₂HPO₄.12H₂O in 2974 mg of distilled water.

### TEST EXAMPLES

### Test Example 1

### Assay for Inhibitory Activity Against Kinase

(7S,12S,13R,Z)-4,12,13-trihydroxy-2-methoxy-7-methyl-7,8,13,14-tetrahydro-5H-dibenzo[c,e][1]oxacyclotetradecen-5,11(12H)-dione (Compound 24), representative of the compounds of the present invention, was assayed for inhibitory activity against a broad spectrum of protein kinases, and the results are expressed as % inhibition in Table 1, below.

TABLE 1

Cpd. 24

| Kinase | IC₅₀ (M) | % Inhibition (1 uM) |
|---|---|---|
| FLT3 (D835Y) | 2.14E−07 | |
| FLT3 (ITD) | 5.22E−07 | |
| FLT3 | | 95 |
| FLT1/VEGFR1 | | 64 |
| FLT4/VEGFR3 | | 97 |
| PDGFRa | | 79 |
| PDGFRb | | 70 |
| PDGFRa (D842V) | 1.97E−07 | |
| PDGFRa (T674I) | 2.21E−06 | |
| PDGFRa (V561D) | 1.87E−07 | |

### Test Example 2

### Assay for Suppressive Activity Against Growth of AML (Acute Myeloid Leukemia) Cells

The MV4-11 cell line was grown at 37° C. in RPMI medium [Gibco, New York, USA/10% FBS (Gibco), containing 1% penicillin/streptomycin (Gibco)] in a 5% CO₂ atmosphere. The grown MV4-11 cell line was withdrawn into PBS, seeded at a density of 3×10⁴ cells/well into 96-well plates, and cultured at 37° C. for 24 hrs in a 5% CO₂ atmosphere. Various concentrations of the compound (8 points) were prepared by serial dilution in 10 mM DMSO, and the MV4-11 cell line was treated with various concentrations of the compound for 48 hrs. To assess cell viability, 3-(4,5-dimethylthiazol-2-yl)-2,5-diphenyl tetrazolium bromide (MTT) assay (CellTiter 96 Assay, Promega) was performed.

Briefly, the dye was added in an amount of 15 μL to each well and incubated for 2 hrs. Then, each well was incubated with 100 μL of a stop solution for 24 hrs, followed by reading absorbance at 590 nm on EnVision2103. GI₅₀ was calculated using GraphPad Prism 4.0.

(7S,12S,13R,Z)-4,12,13-trihydroxy-2-methoxy-7-methyl-7,8,13,14-tetrahydro-5H-dibenzo[c,e][1]oxacyclotetradecen-5,11(12H)-dione (Compound 24), representative of the compounds of the present invention, was assayed for suppressive activity against the growth of AML (acute myeloid leukemia) cells, and the results are summarized in Table 2, below.

TABLE 2

| Cancer Cell | Cpd. 24 |
|---|---|
| Line | GI₅₀ (μM) |
| Molm-14 | 0.09 |
| MV4-11 | 0.03 |

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A compound, selected from among a resorcyclic acid lactone compound represented by the following Chemical Formula F, and a pharmaceutically acceptable salt thereof: 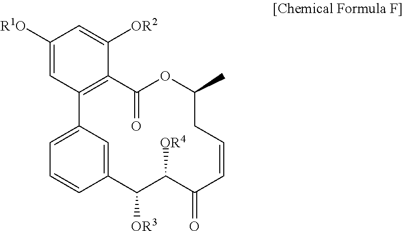
(wherein R¹, R², R³, and R⁴ independently represent a hydrogen atom or C1˜C10 alkyl).

2. The compound of claim 1, being in an optically pure form or as a racemate.

3. The compound of claim 2, wherein R¹ is C1˜C10 alkyl, and R³ and R⁴ are each a hydrogen atom.

4. The compound of claim 3, being (7S,12S,13R,Z)-4,12,13-trihydroxy-2-methoxy-7-methyl-7,8,13,14-tetrahydro-5H-dibenzo[c,e][1]oxacyclotetradecen-5,11(12H)-dione.

5. An anti-cancer composition comprising the compound defined in claim 1.

6. The anti-cancer composition of claim 5, having inhibitory activity against a kinase selected from the group consisting of FLT3 (D835Y), FLT3 (ITD), FLT3, FLT1/VEGFR1, FLT4/VEGFR3, PDGFRa, PDGFRb, PDGFRa (D842V), PDGFRa (T674I), and PDGFRa (V561 D).

7. The anti-cancer composition of claim 5 for use in treatment and prevention of blood cancer.

8. The anti-cancer composition of claim 7, wherein the blood cancer is acute myeloid leukemia (AML).

9. A method for synthesizing a resorcyclic acid lactone compound, as illustrated in the following Reaction Scheme, comprising:
converting compound into a lactam compound through hydrolysis and Mitsunobu reaction;
removing p-methoxybenzyl (PMB) from the compound by deprotection to give an alcohol compound;
oxidizing compound into an α,β-unsaturated ketone compound in presence of Dess-Martin periodinane and a base;
performing a deprotection reaction to remove methoxymethyl (MOM) from compound to afford resorcyclic acid lactone compound. 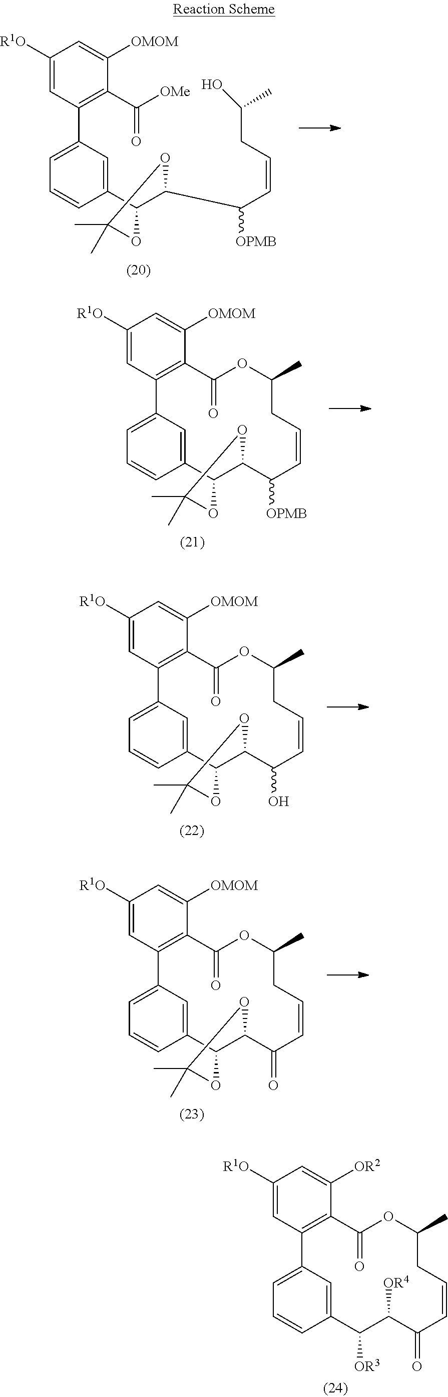
(wherein R¹, R², R³, and R⁴ independently represent a hydrogen atom or C1˜C10 alkyl, MOM represents methoxymethyl, and PMB represents p-methoxybenzyl)

10. An intermediate compound for use in synthesis of the resorcyclic acid lactone compound of claim 1, selected from the group consisting of:
5-(3-(hydroxymethyl)phenyl)-7-methoxy-2,2-dimethyl-4H-benzo[d][1,3]dioxin-4-one);
3-(7-methoxy-2,2-dimethyl-4-oxo-4H-benzo[d][1,3]dioxin-5-yl)benzaldehyde;
(E)-methyl 3-(3-(7-methoxy-2,2-dimethyl-4-oxo-4H-benzo[d][1,3]dioxin-5-yl)phenyl)acrylate;
(2S,3R)-methyl 2,3-dihydroxy-3-(3-(7-methoxy-2,2-dimethyl-4-oxo-4H-benzo[d][1,3]dioxin-5-yl)phenyl)propanoate;
(4S,5R)-methyl 5-(3-(7-methoxy-2,2-dimethyl-4-oxo-4H-benzo[d][1,3]dioxin-5-yl)phenyl)-2,2-dimethyl-1,3-dioxorane-4-carboxylate;
(4S,5R)-methyl 5-(3′-hydroxy-5′-methoxy-2′-(methoxycarbonyl)-[1,1′-biphenyl]-3-yl)-2,2-dimethyl-1,3-dioxorane-4-carboxylate;
(4S,5R)-methyl 5-(5′-methoxy-2′-(methoxycarbonyl)-3′-(methoxymethoxy)-[1,1′-biphenyl]-3-yl)-2,2-dimethyl-1,3-dioxorane-4-carboxylate;
methyl 3′-((4R,5R)-5-(hydroxymethyl)-2,2-dimethyl-1,3-dioxoran-4-yl)-5-methoxy-3-(methoxymethoxy)-[1,1′-biphenyl]-2-carboxylate;
methyl 3′-(4R,5R)-5-((5R)-5-(tert-butyldiphenylsily)oxy)-1-hydroxyhex-2-yn-1-yl)-2,2-dimethyl-1,3-dioxoran-4-yl)-5-methoxy-3-(methoxymethoxy)[1,1′-biphenyl]-2-carboxylate;
methyl 3′-(4R,5R)-5-((5R)-5-((tert-butyldimethylsily)oxy)-1-hydroxyhex-2-yn-1-yl)-2,2-dimethyl-1,3-dioxoran-4-yl)-5-methoxy-3-(methoxymethoxy)-[1,1′-biphenyl]-2-carboxylate;
methyl 3′-((4R,5R)-5-((5R,Z)-5-((tert-butyldiphenylsily)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate;
methyl 3′-((4R,5R)-5-((5R,Z)-5-((tert-butyldimethylsilyl)oxy)-1-hydroxyhex-2-en-1-yl)-2,2-dimethyl-1,3-dioxoran-4-yl)-5-methoxy-3-(methoxymethoxy)-[1,1′-biphenyl]-2-carboxylate;
methyl 3′-((4R,5R)-5-((5R,Z)-5-((tert-butyldiphenylsily)oxy)-1-((4-methoxybenzyl)oxy)hex-2-en-1-yl)-2,2-dimethyl-1,4-dioxoran-4-yl)-5-methoxy-3-(methoxymethoxy)-[1,1′-biphenyl]-2-carboxylate;
methyl 3′-((4R,5R)-5-((5R,Z)-5-((tert-butyldimethylsilyl)oxy)-1-((4-methoxy benzyl)oxy)hex-2-en-2-yl)-2,2-dimethyl-1,3-dioxoran-4-yl)-5-methoxy-3-(methoxymethoxy)-[1,1′-biphenyl]-2-carboxylate;
methyl 3′-((4R,5R)-5-((5R,Z)-5-hydroxy-1-((4-methoxybenzyl)oxy)hex-2-en-1-yl)-2,2-dimethyl-1,3-dioxoran-4-yl)-5-methoxy-3-(methoxymethoxy)-[1,1′-biphenyl]-2-carboxylate;
(3aR,8S,19aR,Z)-13-methoxy-4-((4-methoxybenzyl)oxy)-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one;
(3aR,8S,19aR,Z)-4-hydroxy-13-methoxy-11-(methoxymethoxy)-2,2,8-trimethyl-7,8,19,19a-tetrahydro-3aH-dibenzo[c,e][1,3]dioxolo[4,5-h][1]oxacyclotetradecen-10(4H)-one; and
(3aR,8S,19aR,Z)-13-methoxy-11-(methoxymethoxy)-2,2,8-trimethyl-7,8,19,19a-tetrahydro-3aH-dibenzo[c,e][1.3]dioxolo[4,5-h][1]oxacyclotetradecen-4,10-dione.

11. An anti-cancer composition comprising the compound defined in claim 2.

12. An anti-cancer composition comprising the compound defined in claim 3.

13. An anti-cancer composition comprising the compound defined in claim 4.
